(19) United States
(12) Patent Application Publication
(10) Pub. No.: US 2012/0064110 A1
(43) Pub. Date: Mar. 15, 2012

(54) SYNTHETIC NANOCARRIER VACCINES COMPRISING PEPTIDES OBTAINED OR DERIVED FROM HUMAN INFLUENZA A VIRUS HEMAGGLUTININ

(75) Inventors: Petr Ilyinskii, Cambridge, MA (US); Yun Gao, Southborough, MA (US); Fen-ni Fu, Northborough, MA (US); Grayson B. Lipford, Watertown, MA (US)

(73) Assignee: Selecta Biosciences, Inc., Watertown, MA (US)

(21) Appl. No.: 13/213,496

(22) Filed: Aug. 19, 2011

Related U.S. Application Data
(60) Provisional application No. 61/375,543, filed on Aug. 20, 2010.
(60) Provisional application No. 61/375,635, filed on Aug. 20, 2010.
(60) Provisional application No. 61/375,586, filed on Aug. 20, 2010.

(51) Int. Cl. A61K 39/385; A61P 31/16; B32B 5/16; C07K 14/11; C07K 9/00; B82Y 5/00

(52) U.S. Cl. 424/196.11; 530/322; 428/402; 977/773; 977/762; 977/738; 977/917

(57) ABSTRACT

This invention relates to compositions and methods that can be used immunize a subject against influenza. Generally, the compositions and methods include peptides obtained or derived from human influenza A virus hemagglutinin.

### RELATED APPLICATIONS

This application claims the benefit under 35 U.S.C. §119 of U.S. provisional applications 61/375,586, 61/375,635, and 61/375,543, each filed Aug. 20, 2010, the entire contents of each of which are incorporated herein by reference.

### FIELD OF THE INVENTION

This invention relates to compositions and methods that can be used immunize a subject against influenza. Generally, the compositions and methods include peptides obtained or derived from human influenza A virus hemagglutinin.

### BACKGROUND OF THE INVENTION

Influenza is an infectious disease caused by RNA viruses of the family Orthomyxoviridae. Common symptoms of the disease include chills, fever, sore throat, muscle pains, severe headache, coughing, and fatigue. In more serious cases, influenza can lead to pneumonia, which can be fatal. Influenza spreads around the world in seasonal epidemics, resulting in the deaths of between 250,000 and 500,000 people every year, and up to millions in some pandemic years. Human influenza A virus (“HIAV”) is the most common strain of the virus, and is responsible for all major influenza pandemics.

Major human influenza viral envelope component hemagglutinin (HA) is a surface influenza glycoprotein. HA is known to induce protective immune responses that can efficiently prevent viral infection and/or virus-induced disease in animal models and human subjects (Ellebedy and Webby, 2009; Roose et al., 2009).

HA exists in nature as a trimer composed of three identical monomers assembled into a central-helical coiled coil that consists of stem (stalk) region and three globular domains. The three globular domains contain binding sites for cell surface receptors that are essential for virus attachment to the target cell. The HA monomers are composed of two disulfide-linked chains, HA1 and HA2, which are created by proteolytic cleavage of the precursor HA0 during viral maturation. Aside from cell attachment, HA plays an essential role in infection by initiating a pH-dependent fusion of viral and endosomal membranes upon endocytosis. This fusion process induces dramatic conformational changes in HA2, which involve translocations of several HA2 domains, exposure of fusion peptides and formation of hydrophobic bonds between HA and the target membrane (Cross et al. 2009; Isin et al., 2002; Ekiert et al., 2009).

HA is known to induce strong antibody-mediated immune responses against influenza virus and is a central component of many influenza vaccines. However, utilization of HA-based vaccines is plagued with two well-known problems. These inherent issues undercutting HA-based immunization schemes are closely related to HIAV biology. HIAV possesses an ability to constantly acquire new structural mutations and thus change antigenically (antigenic drift). HIAV also possesses a capacity for gene exchange and recombination, which often results in generation of a viral strain with a completely novel surface gene composition (antigenic shift).

Continuous antigenic changes of HIAV necessitate seasonal construction of influenza vaccine de novo utilizing those HA protein that are carried by the viral strains predicted to cause epidemics during the next season. This approach is prone to mistakes as during 2007-2008 epidemics, when two of the three vaccines prepared early in the year failed to target those viral strains that actually emerged. Moreover, it requires repeated manufacturing of high amounts of vaccine containing different HAs, which sometimes (as during the most recent 2009-2010 season) may not be accomplished timely to provide sufficient vaccination material for the general population.

Constant changes in HA leads to accumulation of mutations in its dominant antigenic epitopes, which manifestly contributes to the non-stop waning of anti-HA immunity in a vaccinated population. These epitopes are mostly localized in HA variable globular regions, which are easily accessible to antibodies and are being targeted by humoral response in the majority of vaccinated individuals or animals (Caton et al., 1982; Kaverin et al., 2002; Tsuchiya et al., 2001; Wiley et al. 1981). Thus, HAs of new viral strains continuously emerging by antigenic drift are recognized less efficiently, which leads to a constant decrease of protection in a vaccinated population. Moreover, current HIAV vaccines won't protect against already existing viral strains that carry HA types unrelated to those used for vaccination. Furthermore, HA-directed immunity may be essentially ineffective against a completely novel HIAV strain, emerging as a result of antigenic shift.

### SUMMARY OF THE INVENTION

In one aspect, a dosage form comprising synthetic nanocarriers coupled to peptides that are obtained or derived from human influenza A virus hemagglutinin is provided. In one embodiment, the dosage form further comprises a pharmaceutically acceptable excipient. In another embodiment, the peptides are obtained or derived from an HA1 subunit of human influenza A virus hemagglutinin. In still another embodiment, the peptides are obtained or derived from an HA2 subunit of human influenza A virus hemagglutinin. In yet another embodiment, the peptides are obtained or derived from an A-helix of an epitope on HA2 subunit of human influenza A virus hemagglutinin that is bound by antibody CR6261.

In another embodiment, the peptides obtained or derived from human influenza A virus hemagglutinin comprise a peptide with the amino acid sequence as set forth in or a peptide as set forth in the following formula:

(SEQ ID NO: 1)
Acetyl-X₁KE X₂QKAID X₃TN X₄VN X₅I X₆ X₇-R

where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10), and
R═COOH or a linking group for coupling to the synthetic nanocarriers.

In one embodiment, the peptides comprise a peptide with an amino acid sequence as set forth in any one of SEQ ID NOs: 1, 11-25 and 27-34. In another embodiment, the peptide comprises Acetyl-Ala-Ala-Asp-Lys-Glu-Ser-Thr-Gln-Lys-Ala-Ile-Asp-Gly-Val-Thr-Asn-Lys-Val-Asn-Ser-Ile-Ile-Asp-Lys-Gly-Gly-NHCH2CCH (C-terminal glycine propargyl amide) (SEQ ID NO: 27). In yet another embodiment, the peptide comprises

Acetyl-Ala-Ala-Asp-Lys-Ala-Ser-Thr-Gln-Ala-Ala-Ile-Asp-Gly-Ala-Thr-Asn-Ala-Val-Asn-Ser-Ala-Ile-Glu-Ala-Gly-Gly-NHCH2CCH (C-terminal glycine propargyl amide) (SEQ ID NO: 28). In still another embodiment, the peptide comprises Acetyl-AADAADKEAAQKAIDAATNAVNAAIEAANAAGG-NHCH₂CCH (C-terminal glycine propargyl amide) (SEQ ID NO: 29). In yet another embodiment, the peptide comprises Acetyl-AADAADKEAAQKALDAATNALNAAIEAANAAGG-NHCH₂CCH (C-terminal glycine propargyl amide) (SEQ ID NO: 30). In a further embodiment, the peptide comprises Acetyl-AADAADKEAKQKAIDAATNAVNSAIEAANKAGG-NHCH₂CCH (C-terminal glycine propargyl amide) (SEQ ID NO: 31). In yet a further embodiment, the peptide comprises Acetyl-ILLAADKEAAQKALDAATNALNAAIEAANALLI-NHCH₂CCH (C-terminal glycine propargyl amide) (SEQ ID NO: 32).

In another embodiment, the peptides coupled to the synthetic nanocarriers comprise a peptide with any of the amino acid sequences provided herein. In yet another embodiment, the peptides coupled to the synthetic nanocarriers comprise any of the peptides provided herein. In some embodiments, the peptides coupled to the synthetic nanocarriers are of the same type (i.e., are identical). In other embodiments, two or more types of peptides are coupled to the synthetic nanocarriers. In still other embodiments, at least a portion of the peptides are coupled to a surface of the synthetic nanocarriers. In one embodiment, the coupling is non-covalent coupling. In another embodiment, the coupling is covalent coupling.

In one embodiment, the synthetic nanocarriers are further coupled to one or more adjuvants. In another embodiment, the one or more adjuvants comprise Pluronic® block co-polymers, specifically modified or prepared peptides, stimulators or agonists of pattern recognition receptors, mineral salts, alum, alum combined with monphosphoryl lipid (MPL) A of Enterobacteria, MPL® (ASO4), saponins, QS-21,Quil-A, ISCOMs, ISCOMATRIX™, MF59™, Montanide® ISA 51, Montanide® ISA 720, AS02, liposomes and liposomal formulations, AS01, synthesized or specifically prepared microparticles and microcarriers, bacteria-derived outer membrane vesicles of N. gonorrheae or Chlamydia trachomatis, chitosan particles, depot-forming agents, muramyl dipeptide, aminoalkyl glucosaminide 4-phosphates, RC529, bacterial toxoids, toxin fragments, agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; type I interferons; poly I:C; bacterial lipopolysacccharide (LPS); VSV-G; HMGB-1; flagellin or portions or derivatives thereof; or immunostimulatory DNA molecules comprising CpGs, agonists for DC surface molecule CD40; type I interferons; poly I:C12U; bacterial lipopolysacccharide (LPS); VSV-G; HMGB-1; flagellin or portions or derivatives thereof; immunostimulatory DNA molecules comprising CpGs; proinflammatory stimuli released from necrotic cells; urate crystals; activated components of the complement cascade; activated components of immune complexes; complement receptor agonists; cytokines; or cytokine receptor agonists. In yet another embodiment, the one or more adjuvants comprise agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines, imiquimod, resiquimod, immunostimulatory DNA molecules comprising CpGs, poly I:C, or poly I:C12U.

In one embodiment, the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles. In another embodiment, the synthetic nanocarriers comprise poly(lactic acid)-polyethyleneglycol copolymer, poly(glycolic acid)-polyethyleneglycol copolymer, or poly(lactic-co-glycolic acid)-polyethyleneglycol copolymer.

In still another embodiment, the synthetic nanocarriers are further coupled to T-helper antigens. In one embodiment, the T-helper antigen comprises any of the T-helper antigens provided herein. In another embodiment, the amino acid sequence of the T-helper antigen comprises the amino acid sequence as set forth in SEQ ID NO: 26.

In yet another embodiment, the synthetic nanocarriers are present in an amount effective to provide an immune response to the peptides when the synthetic nanocarriers or dosage form are/is administered to a subject.

In still another embodiment, the dosage form further comprises influenza antigen that is not coupled to the synthetic nanocarriers.

In another aspect, a dosage form comprising peptides obtained or derived from human influenza A virus hemagglutinin that generates in a subject polyclonal antibodies that compete for binding to human influenza A virus hemagglutinin with a control antibody, wherein the control antibody is CR6261 is provided. In one embodiment, whether or not the polyclonal antibodies compete for binding is assessed with any of the methods described herein. In another embodiment, the competitive binding is assessed using the entire human influenza A virus hemagglutinin. In yet another embodiment, the competitive binding is assessed using the entire HA1 subunit of human influenza A virus hemagglutinin. In still another embodiment, the competitive binding is assessed using the entire HA2 subunit of human influenza A virus hemagglutinin. In yet another embodiment, the competitive binding is assessed using the A-helix of an epitope on HA2 subunit of human influenza A virus hemagglutinin that is bound by antibody CR6261. In a further embodiment, the competitive binding is assessed using a peptide with the amino acid sequence as set forth in or a peptide as set forth in the following formula:

(SEQ ID NO: 1)
Acetyl-X₁KE X₂QKAID X₃TN X₄VN X₅I X₆ X₇-R

where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10), and
R═COOH or a linking group. In one embodiment, the competitive binding is assessed using a peptide with an amino acid sequence as set forth in any one of SEQ ID NOs: 1, 11-25 and 27-34. In still another embodiment, the competitive binding is assessed using any of the peptides provided herein.

In one embodiment, the dosage form further comprises a pharmaceutically acceptable excipient.

In another embodiment, the peptides are obtained or derived from an HA1 subunit of human influenza A virus hemagglutinin. In still another embodiment, the peptides are obtained or derived from an HA2 subunit of human influenza A virus hemagglutinin. In yet another embodiment, the peptides are obtained or derived from an A-helix of an epitope on HA2 subunit of human influenza A virus hemagglutinin that is bound by antibody CR6261.

In another embodiment, the peptides obtained or derived from human influenza A virus hemagglutinin comprise a peptide with the amino acid sequence as set forth in or a peptide as set forth in the following formula:

(SEQ ID NO: 1)
Acetyl-X₁KE X₂QKAID X₃TN X₄VN X₅I X₆ X₇-R

where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10), and
R═COOH or a linking group.

In one embodiment, the peptides comprise a peptide with an amino acid sequence as set forth in any one of SEQ ID NOs: 1, 11-25 and 27-34. In another embodiment, the peptide comprises Acetyl-Ala-Ala-Asp-Lys-Glu-Ser-Thr-Gln-Lys-Ala-Ile-Asp-Gly-Val-Thr-Asn-Lys-Val-Asn-Ser-Ile-Ile-Asp-Lys-Gly-Gly-NHCH2CCH (C-terminal glycine propargyl amide) (SEQ ID NO: 27). In yet another embodiment, the peptide comprises Acetyl-Ala-Ala-Asp-Lys-Ala-Ser-Thr-Gln-Ala-Ala-Ile-Asp-Gly-Ala-Thr-Asn-Ala-Val-Asn-Ser-Ala-Ile-Glu-Ala-Gly-Gly-NHCH2CCH (C-terminal glycine propargyl amide) (SEQ ID NO: 28). In still another embodiment, the peptide comprises Acetyl-AADAADKEAAQKAIDAATNAVNAAIEAANAAGG-NHCH₂CCH (C-terminal glycine propargyl amide) (SEQ ID NO: 29). In yet another embodiment, the peptide comprises Acetyl-AADAADKEAAQKALDAATNALNAAIEAANAAGG-NHCH₂CCH (C-terminal glycine propargyl amide) (SEQ ID NO: 30). In a further embodiment, the peptide comprises Acetyl-AADAADKEAKQKAIDAATNAVNSAIEAANKAGG-NHCH₂CCH (C-terminal glycine propargyl amide) (SEQ ID NO: 31). In yet a further embodiment, the peptide comprises Acetyl-ILLAADKEAAQKALDAATNALNAAIEAANALLI-NHCH₂CCH (C-terminal glycine propargyl amide) (SEQ ID NO: 32).

In another embodiment, the peptides comprise a peptide with any of the amino acid sequences provided herein. In yet another embodiment, the peptides comprise any of the peptides provided herein. In some embodiments, the peptides in the dosage form are of the same type (i.e., are identical). In other embodiments, two or more types of peptides are comprised in the dosage form.

In another embodiment, the dosage form further comprises one or more adjuvants. In one embodiment, the one or more adjuvants comprise Pluronic® block co-polymers, specifically modified or prepared peptides, stimulators or agonists of pattern recognition receptors, mineral salts, alum, alum combined with monphosphoryl lipid (MPL) A of Enterobacteria, MPL® (ASO4), saponins, QS-21,Quil-A, ISCOMs, ISCOMATRIX™, MF59™, Montanide® ISA 51, Montanide® ISA 720, AS02, liposomes and liposomal formulations, AS01, synthesized or specifically prepared microparticles and microcarriers, bacteria-derived outer membrane vesicles of N. gonorrheae or Chlamydia trachomatis, chitosan particles, depot-forming agents, muramyl dipeptide, aminoalkyl glucosaminide 4-phosphates, RC529, bacterial toxoids, toxin fragments, agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines; imiquimod; resiquimod; type I interferons; poly I:C; bacterial lipopolysacccharide (LPS); VSV-G; HMGB-1; flagellin or portions or derivatives thereof; or immunostimulatory DNA molecules comprising CpGs, agonists for DC surface molecule CD40; type I interferons; poly I:C12U; bacterial lipopolysacccharide (LPS); VSV-G; HMGB-1; flagellin or portions or derivatives thereof; immunostimulatory DNA molecules comprising CpGs; proinflammatory stimuli released from necrotic cells; urate crystals; activated components of the complement cascade; activated components of immune complexes; complement receptor agonists; cytokines; or cytokine receptor agonists. In another embodiment, the one or more adjuvants comprise agonists of Toll-Like Receptors 2, 3, 4, 5, 7, 8, 9 and/or combinations thereof; adenine derivatives; immunostimulatory DNA; immunostimulatory RNA; imidazoquinoline amines, imidazopyridine amines, 6,7-fused cycloalkylimidazopyridine amines, 1,2-bridged imidazoquinoline amines, imiquimod, resiquimod, immunostimulatory DNA molecules comprising CpGs, poly I:C, or poly I:C12U.

In some embodiments, the dosage form further comprises T-helper antigens. In one embodiment, the T-helper antigen comprises any of the T-helper antigens provided herein. In another embodiment, the amino acid sequence of the T-helper antigen comprises the amino acid sequence as set forth in SEQ ID NO: 26.

In a further embodiment, the dosage form further comprises a carrier that boosts an immune response to the peptides when administered to a subject. In one embodiment, the peptides are coupled to the carrier. In another embodiment, the carrier comprises keyhole limpet hemocyanin, concholepas concholepas hemocyanin, bovine serum albumin, cationized BSA or ovalbumin. In yet another embodiment, the carrier comprises a synthetic nanocarrier. In still another embodiment, a linking group couples the peptides to the carrier. In another embodiment, the carrier is also coupled to T-helper antigens. In one embodiment, the T-helper antigen comprises any of the T-helper antigens provided herein. In another embodiment, the amino acid sequence of the T-helper antigen comprises the amino acid sequence as set forth in SEQ ID NO: 26. In still another embodiment, the carrier is also coupled to one or more adjuvants.

In one embodiment, the synthetic nanocarrier comprises a/an lipid-based nanoparticle, polymeric nanoparticle, metallic nanoparticle, surfactant-based emulsion, dendrimer, buckyball, nanowires, virus-like particle, peptide or protein-based particle, lipid-polymer nanoparticle, spheroidal nanoparticle, cubic nanoparticle, pyramidal nanoparticle, oblong nanoparticle, cylindrical nanoparticle, or toroidal nanoparticle. In another embodiment, the synthetic nanocarrier comprises poly(lactic acid)-polyethyleneglycol copolymer, poly(glycolic acid)-polyethyleneglycol copolymer, or poly(lactic-co-glycolic acid)-polyethyleneglycol copolymer.

In still another embodiment, the peptides, synthetic nanocarriers or the dosage forms is/are in an amount effective to provide an immune response to the peptides when administered to a subject.

In yet another embodiment, the dosage form further comprises influenza antigen. In another embodiment, when the dosage form comprises a carrier, the influenza antigen is not coupled to the carrier. In one embodiment, the carrier is a synthetic nanocarrier.

In another aspect, a method comprising administering any of the dosage forms to a subject is provided. In one embodiment, the dosage form is administered at least once to the subject. In another embodiment, the dosage form is administered at least twice to the subject. In yet another embodiment, the dosage form is administered at least three times to the subject. In still another embodiment, the dosage form is administered at least four times to the subject.

In yet another aspect, a method comprising providing synthetic nanocarriers, and coupling peptides that are obtained or derived from human influenza A virus hemagglutinin to the synthetic nanocarriers is provided. In one embodiment, the coupling comprises covalently coupling the peptides to the synthetic nanocarriers. In another embodiment, the peptides comprise a peptide with any of the amino acid sequences provided herein. In yet another embodiment, the peptides comprise any of the peptides provided herein. In some embodiments, the peptides are of the same type (i.e., are identical). In other embodiments, the peptides are of two or more types.

In still another aspect, a composition, dosage form or vaccine obtained, or obtainable, by any of the methods provided is provided.

In a further aspect, a process for producing a composition, dosage form or vaccine comprising the steps of providing synthetic nanocarriers, and coupling peptides that are obtained or derived from human influenza A virus hemagglutinin to the synthetic nanocarriers is provided. In one embodiment, the coupling comprises covalently coupling the peptides to the synthetic nanocarriers. In another embodiment, the peptides comprise a peptide with any of the amino acid sequences provided herein. In yet another embodiment, the peptides comprise any of the peptides provided herein. In some embodiments, the peptides are of the same type (i.e., are identical). In other embodiments, two or more types of peptides are comprised in the composition, dosage form or vaccine.

In yet a further aspect, a composition comprising a peptide with the amino acid sequence as set forth in or a peptide as set forth in the following formula is provided:

(SEQ ID NO: 1)
Acetyl-X₁KE X₂QKAID X₃TN X₄VN X₅I X₆ X₇-R

where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10)
R═COOH or a linking group.

In one embodiment, a composition comprising a peptide that has the amino acid sequence as set forth in SEQ ID NO: 1 is provided.

In another aspect, any of the dosage forms or compositions provided may be for use in therapy or prophylaxis.

In yet another aspect, any of the dosage forms or compositions provided may be for use in any of the methods provided.

In still another aspect, any of the dosage forms or compositions provided may be for use in vaccination.

In a further aspect, any of the dosage forms or compositions provided may be for use in a method of therapy or prophylaxis of influenza virus infection, for example influenza A virus infection.

In yet a further aspect, any of the dosage forms or compositions provided may be for use in a method of therapy or prophylaxis comprising administration by a subcutaneous, intramuscular, intradermal, oral, intranasal, transmucosal, sublingual, rectal, ophthalmic, transdermal, transcutaneous route or by a combination of these routes.

In another aspect, any of the dosage forms or compositions provided may be for the manufacture of a medicament, for example a vaccine, for use in any of the methods provided.

### BRIEF DESCRIPTION OF THE DRAWINGS

FIG. 1 provides circular dichroic measurements of peptides.

FIG. 2 provides circular dichroic measurements of additional peptides.

FIG. 3 provides anti-HAP antibody titers after NC-HAP vaccination. Groups 1 and 2: immunized with NC-HAP1 or NC-HAP2, group 3: immunized with purified HA protein; group 4: immunized with purified HA in alum.

FIG. 4 provides anti-HAP antibody titers after NC-HAP vaccination. Groups 1-5: immunized with NC-HAP54.1, NC-HAP5, NC-HAP54.4, NC-HAP55.32.5, or NC-HAP2, respectively; group 6: immunized with purified HA protein in alum.

FIG. 5 provides anti-HAP antibody titers after NC-HAP vaccination. Groups 1-5: immunized with NC-HAP54.1, NC-HAP5, NC-HAP54.4, NC-HAP55.32.5, or NC-HAP2, respectively; group 6: immunized with 10 μg of purified HA protein in alum (1:1). Titers determined by ELISA against HAP54.1, HAP5, HAP54.4, HAP55.32.5, or HAP2. Titers for day 39 after the first immunization are shown.

FIG. 6 provides anti-H5N1 HA protein antibody titers after NC-HAP vaccination (5 animals/group, subcutaneous route, injected 3 times with a 2 week interval and once at day 115). Groups 1-5: immunized with 100 μg of NC-HAP54.1, NC-HAP5, NC-HAP54.4, NC-HAP55.32.5, or NC-HAP2, respectively; group 6: immunized with 10 μg of purified HA protein in alum (1:1). Titers determined by ELISA against influenza virus H5N1 hemagglutinin protein. Titers for day 39 after the first immunization are shown.

### DETAILED DESCRIPTION OF THE INVENTION

### I. Introduction

Significant investment of public and medical efforts is necessary for targeting HIAV, encompassing constant epidemiologic surveillance of influenza and annual re-vaccination of susceptible populations with vaccines carrying different HA subtypes. A vaccination approach enabling the circumvention of HA variability and providing for long-term and broad-spectrum immunity against influenza will be greatly beneficial. Therefore, what is needed are compositions and methods that could address the problems noted above that are associated with producing vaccines against human influenza A virus.

The inventors have unexpectedly and surprisingly discovered that the problems and limitations noted above can be overcome by practicing the invention disclosed herein. In particular, the inventors have unexpectedly discovered that it is possible to provide compositions, and related methods, that comprise a dosage form comprising: synthetic nanocarriers coupled to peptides that are obtained or derived from human influenza A virus hemagglutinin. In another embodiment, the inventors have discovered methods comprising providing synthetic nanocarriers; and coupling peptides that are obtained or derived from human influenza A virus hemagglutinin to the synthetic nanocarriers. In still another embodiment, the inventors have discovered a peptide of the formula:

(SEQ ID NO: 1)
Acetyl-X₁KE X₂QKAID X₃TN X₄VN X₅I X₆ X₇-R

where
X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10)
R═COOH, or a linking group for coupling to a carrier such as a synthetic nanocarrier.

In some embodiments, the present invention provides dosage forms and methods that induce strong cross-protective immunity against multiple influenza strains thus providing for a long-term and broad protection against seasonal and pandemic influenza without recurring immunization. Collectively, while several investigative routes to create an effective, cross-protective and easy-to-manufacture influenza vaccine have been actively pursued in the field, none of them as of to-date has solved the issue of immunity waning due to antigenic shift and antigenic drift. This deficiency has led to constant re-vaccination of susceptible population groups coupled with the necessity of perpetual re-composition and re-manufacturing of influenza vaccines. These shortcomings of current influenza vaccination schemes are addressed by the inventive compositions and methods disclosed herein.

HA variability is linked to evolutionary pressure directed against globular HA heads, which are exposed to antibodies. At the same time, other regions of HA necessary for its structural integrity and membrane fusion are both highly conserved and, while antigenic, are less accessible to antibodies. The inventors recognized that a strong antibody response against a highly conserved HA epitope will have protective activity against widely variable influenza strains. This realization led to the recognition that conserved influenza epitopes, if put in the proper immunological context, may be able to generate a broad cross-protective response. Recently, several monoclonal antibodies have been described that are capable of binding and neutralizing widely divergent HA subtypes (Throsby et al., 2008; Sui et al., 2009). Furthermore, binding sites for two of these antibodies have been shown to reside on the stem of the HA2 chain (Ekiert et al., 2009; Sui et al., 2009), although in both cases full neutralizing epitopes were conformational (three-dimensional), thus hindering their utilization in vaccination. However, a conformational epitope bound by CR6261, which is one of these two antibodies, clearly consists of two parts. One of the two parts of the epitope bound by CR6261 is completely located within a linear fragment forming a short alpha helix within the HA2 subunit of human influenza A virus hemagglutinin (Ekiert et al., 2009) that is termed “A-helix” according to the nomenclature proposed by Bullough et al., Nature. 1994;371:37. This A-helix is translocated during HA-mediated membrane fusion and is likely to play an important role in this process. Moreover, the linear A-helix component of the CR6261 neutralizing epitope was shown to form most of the contacts with the antibody. Furthermore, CR6261 abolished pH-mediated conformational changes of HA in vitro.

Prior to the present invention, it was not obvious that a short peptide sequence might be utilized for efficient vaccination since peptide-based vaccines are known to be weakly immunogenic in many systems (Black et al., 2010; Purcell et al., 2003). Based on the work presented herein, however, it is believed that antibodies generated against a linear peptide mimicking the viral epitope contained within A-helix of HA2 can neutralize widely divergent strains of HIAV.

In the embodiments illustrated by Examples 1, 2, and 3, antigenic peptides were covalently coupled to the synthetic nanocarriers (termed “HAP-NC”). The resulting dosage forms are completely synthetic and thus non-infectious and easy to manufacture. Example 4 illustrates a non-covalent coupling between inventive peptides and synthetic nanocarriers to exemplify an embodiment of the present invention.

Example 5 illustrates that covalent coupling of several viral antigens to polymeric synthetic PLA/PLGA-based synthetic nanocarrier (NC) via a PLA-PEG linker have been achieved and that such NC-coupled viral antigens are immunogenic in vivo. These antigens include modified HA-related peptides (HAP-1 and HAP-2) mimicking the conserved antigenic epitope based on the highly pathogenic strain A/Vietnam/1203/04(H5N1) HA2 stem-forming A-helix residues 35-58. Additionally, the inventive synthetic nanocarriers contain TLR7/8 agonist resiquimod (R848) which has been covalently coupled to PLGA, and an ovalbumin peptide as a T-helper antigen. As the results of Example 5 illustrate, immunization with the inventive synthetic nanocarriers coupled with HA2-based peptides resulted in efficient and cross-reactive immune responses to influenza HA.

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to a “DNA molecule” includes a mixture of two or more such DNA molecules or a plurality of such DNA molecules, reference to “an adjuvant” includes a mixture of two or more such materials or a plurality of adjuvant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

The invention will be described in more detail below.

### II. Definitions

“Adjuvant” means an agent that does not constitute a specific antigen, but boosts the strength and longevity of immune response to a concomitantly administered antigen. Such adjuvants may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (ASO4), MPL A of above-mentioned bacteria separately, saponins, such as QS-21,Quil-A, ISCOMs, ISCOMATRIX™, emulsions such as MF59™, Montanide® ISA 51 and ISA 720, AS02 (QS21+squalene+MPL®) , liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In embodiments, adjuvants comprise agonists for pattern recognition receptors (PRR), including, but not limited to Toll-Like Receptors (TLRs), specifically TLRs 2, 3, 4, 5, 7, 8, 9 and/or combinations thereof. In other embodiments, adjuvants comprise agonists for Toll-Like Receptors 3, agonists for Toll-Like Receptors 7 and 8, or agonists for Toll-Like Receptor 9; preferably the recited adjuvants comprise imidazoquinolines; such as R848 (also known as resiquimod); adenine derivatives, such as those disclosed in U.S. Pat. No. 6,329,381 (Sumitomo Pharmaceutical Company); US Published Patent Application 2010/0075995 to Biggadike et al., or WO 2010/018132 to Campos et al.; immunostimulatory DNA; or immunostimulatory RNA.

In specific embodiments, synthetic nanocarriers incorporate as adjuvants compounds that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody immune responses.

In embodiments, adjuvants also may comprise immunostimulatory RNA molecules, such as but not limited to dsRNA, poly I:C or poly I:poly C12U (available as Ampligen®, both poly I:C and poly I:poly C12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an adjuvant may be a TLR-4 agonist, such as bacterial lipopolysacccharide (LPS), VSV-G, and/or HMGB-1. In some embodiments, adjuvants may comprise TLR-5 agonists, such as flagellin, or portions or derivatives thereof, including but not limited to those disclosed in U.S. Pat. Nos. 6,130,082, 6,585,980, and 7,192,725.

In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. No. 6,207,646 to Krieg et al.; U.S. Pat. No. 7,223,398 to Tuck et al.; U.S. Pat. No. 7,250,403 to Van Nest et al.; or U.S. Pat. No. 7,566,703 to Krieg et al.

In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

In embodiments, at least a portion of the dose of adjuvant may be coupled to synthetic nanocarriers, preferably, all of the dose of adjuvant is coupled to synthetic nanocarriers. In other embodiments, at least a portion of the dose of the adjuvant is not coupled to the synthetic nanocarriers. In embodiments, the dose of adjuvant comprises two or more types of adjuvants. For instance, and without limitation, adjuvants that act on different TLR receptors may be combined. As an example, in an embodiment a TLR 7/8 agonist may be combined with a TLR 9 agonist. In another embodiment, a TLR 7/8 agonist may be combined with a TLR 4 agonist. In yet another embodiment, a TLR 9 agonist may be combined with a TLR 3 agonist.

“Administering” or “administration” means providing a drug to a subject in a manner that is pharmacologically useful.

“A-helix of an epitope on HA2 subunit of human influenza A virus hemagglutinin that is bound by antibody CR6261” means a helix formed by amino acid residues 38-58 of the HA2 chain and designated an A-helix (per nomenclature established by Bullough et al., Nature; 371:37, 1994, and used by Ekiert et al., Science; 324:246-51, 2009), the binding surface of which is highly conserved among HIAV subtypes.

“Amount effective” is any amount of a composition provided herein that produces one or more desired immune responses. This amount can be for in vitro or in vivo purposes. For in vivo purposes, the amount can be one that a clinician would believe may have a clinical benefit for a subject at risk of contracting an influenza infection, e.g., a human influenza A virus infection. Amounts effective include amounts that generate a humoral and/or cytotoxic T lymphocyte immune response, or certain levels thereof. An amount that is effective to produce a desired immune responses as provided herein can also be an amount that produces a desired therapeutic endpoint or a desired therapeutic result (e.g., prevents or reduces the severity of influenza infection in a subject).

A subject's immune response can be monitored by routine methods. Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

“Antigen” means a B cell antigen or T cell antigen. In embodiments, antigens are coupled to the synthetic nanocarriers. In other embodiments, antigens are not coupled to the synthetic nanocarriers. In embodiments antigens are coadministered with the synthetic nanocarriers. In other embodiments antigens are not coadministered with the synthetic nanocarriers. “Type(s) of antigens” means molecules that share the same, or substantially the same, antigenic characteristics.

“At least a portion of the dose” means at least some part of the dose, ranging up to including all of the dose.

“B cell antigen” means any antigen that is or recognized by and triggers an immune response in a B cell (e.g., an antigen that is specifically recognized by a B cell receptor on a B cell). In some embodiments, an antigen that is a T cell antigen is also a B cell antigen. In other embodiments, the T cell antigen is not also a B cell antigen.

“Carrier that boosts an immune response to the peptides” means any carrier that when combined with the peptides boosts an immune response against the peptides. Such carriers include, for example, keyhole limpet hemocyanin, concholepas concholepas hemocyanin, bovine serum albumin, cationized BSA or ovalbumin. Such carriers also include synthetic nanocarriers as provided herein. In some embodiments, the peptides are coupled to the carrier. In other embodiments, the peptides are not coupled to the carrier.

“Competes for binding” refers to any competitive inhibition of binding to a target antigen (e.g., inhibition of the binding of a control antibody to a target antigen by polyclonal antibodies generated from the methods or compositions provided herein). Such inhibition can be identified in a simple immunoassay showing the ability of the polyclonal antibodies to block the binding of the control antibody to a target antigen. In some embodiments of such assays, the target antigen is the entire human influenza A virus hemagglutinin protein. In other embodiments, the target antigen is the HA1 subunit of human influenza A virus hemagglutinin. In still other embodiments, the target antigen is the HA2 subunit of human influenza A virus hemagglutinin. In yet other embodiments, the target antigen is the A-helix of an epitope on HA2 subunit of human influenza A virus hemagglutinin that is bound by antibody CR6261. In a further embodiment, the target antigen is a peptide with the amino acid sequence as set forth in or a peptide as set forth in the following formula:

(SEQ ID NO: 1)
Acetyl-X1KE X2QKAID X3TN X4VN X5I X6 X7-R

where X1=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X2=ST or AA
X3=GV or AA
X4=K or A
X5=SI, SA or AA
X6=DK, EA, DA, or AA
X7=GG, or GNG (SEQ ID NO: 10), and
R═COOH. In another embodiment, the target antigen is a peptide comprising an amino acid sequence as set forth in any one of SEQ ID NOs: 1, 11-25 and 27-34. In still another embodiment, the target antigen is any of the peptides provided herein.

Competitive binding is found when the binding of the polyclonal antibodies that are generated with the compositions or methods provided herein inhibit the specific binding of the control antibody, CR6261, to a target antigen, examples of which are provided above. In some embodiments, the polyclonal antibodies inhibit the specific binding of the control antibody by at least 5%, 10%, 15%, 20%, 25%, 30%, 35%, 40%, 45%, 50%, 55%, 60%, 65%, 70%, 75%, 80%, 85%, 90%, 95% or more. Numerous types of competitive binding assays are known, for example: solid phase direct or indirect radioimmunoassay (RIA); solid phase direct or indirect enzyme immunoassay (EIA) sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As an example, such an assay may involve the use of target antigen bound to a solid surface, unlabeled polyclonal antibodies to be tested (in excess) and a labeled control antibody. Competitive inhibition may be measured by determining the amount of label bound to the solid surface in the presence of the control antibody. In an embodiment of such an assay, the polyclonal antibodies are incubated with target antigen bound to a solid surface in the presence of biotinylated control antibody. Following washing, horseradish peroxidase (HRP)-conjugated streptavidin is added to determine the amount of bound biotinylated control antibody. After addition of a substrate (TMB), the development of the enzymatic reaction is stopped with sulfuric acid and absorbance is measured. The percent inhibition is defined relative to the absorbance observed in the presence of an isotype-matched mAb of irrelevant specificity (0% inhibition) and to the absorbance observed using excess test polyclonal antibodies (100% inhibition).

As another example, sera from a subject containing polyclonal antibodies may be obtained. The polyclonal antibodies (unlabeled) are then incubated with target antigen bound to a plate that has been blocked non-specifically with bovine serum albumin or a similar blocking reagent. Unlabeled non-specific antibodies (e.g., those present in sera from a naïve subject or an isotype-matched mAb of irrelevant specificity) are also incubated with the same target antigen bound to another plate that has also been blocked non-specifically with bovine serum albumin or a similar blocking reagent. The plates are then washed and the control antibody labeled with, for example, biotin is added to each. Following washing, horseradish peroxidase (HRP)-conjugated streptavidin is added to determine the amount of bound biotinylated control antibody. After addition of a substrate (TMB), the development of the enzymatic reaction is stopped with sulfuric acid. The absorbance of the two plates is then measured and compared. Any reduction in absorbance is indicative of the level of competitive inhibition.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Concomitantly” means administering two or substances to a subject in a manner that is correlated in time, preferably sufficiently correlated in time so as to provide a modulation in an immune response. In embodiments, concomitant administration may occur through administration of two or more substances in the same dosage form. In other embodiments, concomitant administration may encompass administration of two or more substances in different dosage forms, but within a specified period of time, preferably within 1 month, more preferably within 1 week, still more preferably within 1 day, and even more preferably within 1 hour.

“CR6261” is the monoclonal antibody as described in U.S. Patent Application Publication No. 20090311265.

“Derived” means taken from a source and subjected to substantial modification. For instance, a peptide or nucleic acid with a sequence with only 50% identity to a natural peptide or nucleic acid, preferably a natural consensus peptide or nucleic acid, would be said to be derived from the natural peptide or nucleic acid. Substantial modification is modification that significantly affects the chemical or immunological properties of the material in question. Derived peptides and nucleic acids can also include those with a sequence with greater than 50% identity to a natural peptide or nucleic acid sequence if said derived peptides and nucleic acids have altered chemical or immunological properties as compared to the natural peptide or nucleic acid. These chemical or immunological properties comprise hydrophilicity, stability, affinity, and ability to couple with a carrier such as a synthetic nanocarrier.

“Dosage form” means a pharmacologically and/or immunologically active material in a medium, carrier, vehicle, or device suitable for administration to a subject.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% is exposed to the local environment. Encapsulation is distinct from absorption, which places most or all of a substance on a surface of a synthetic nanocarrier, and leaves the substance exposed to the local environment external to the synthetic nanocarrier.

“HA1 subunit of human influenza A virus hemagglutinin” means the longer (approx. 320-350 amino acids) of two disulfide-linked Human Influenza A virus hemagglutinin (HA) glycoprotein chains formed during HA maturation by proteolytic cleavage of the common HA precursor HA0, and located at the N-terminal part of HA0 (corresponding to 5′-terminal part of full HA gene, encoded by segment 4 of the influenza genome).

“HA2 subunit of human influenza A virus hemagglutinin” means the shorter (approx. 220 amino acids) of two disulfide-linked HA glycoprotein chains formed during HA maturation by proteolytic cleavage of the common HA precursor HA0, located at the C-terminal part of HA0 (corresponding to 3′-terminal part of full HA gene, encoded by segment 4 of the influenza genome).

“Human Influenza A virus hemagglutinin” or “HA” means a major envelope glycoprotein of human A influenza virus encoded by segment 4 of influenza RNA genome. Influenza HA exists in nature as a trimer composed of three identical monomers assembled into a central-helical coiled coil that consists of stem (stalk) region and three globular domains containing binding sites for surface cell receptor, essential for virus attachment to the target cell. These HA monomers are composed of two disulfide-linked glycoprotein chains, the HA1 subunit of human influenza A virus hemagglutinin and the HA2 subunit of human influenza A virus hemagglutinin, which are created by proteolytic cleavage of HA0 precursor during viral maturation. Aside from cell attachment, HA plays an essential role in infection by initiating a pH-dependent fusion of viral and endosomal membranes upon endocytosis. This fusion process induces dramatic conformational changes in HA2, which involve translocations of several HA2 domains, exposure of fusion peptides and formation of hydrophobic bonds between HA and the target membrane (Cross et al. 2009; Isin et al., 2002; Ekiert et al., 2009).

“Isolated nucleic acid” means a nucleic acid that is separated from its native environment and present in sufficient quantity to permit its identification or use. An isolated nucleic acid may be one that is (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. Any of the nucleic acids provided herein may be isolated. Any of the antigens provided herein may be provided as a nucleic acid that encodes it, and such nucleic acid may also be isolated.

“Isolated peptide” means the peptide is separated from its native environment and present in sufficient quantity to permit its identification or use. This means, for example, the peptide may be (i) selectively produced by expression cloning or (ii) purified as by chromatography or electrophoresis. Isolated peptides may be, but need not be, substantially pure. Because an isolated peptide may be admixed with a pharmaceutically acceptable carrier in a pharmaceutical preparation, the peptide may comprise only a small percentage by weight of the preparation. The peptide is nonetheless isolated in that it has been separated from the substances with which it may be associated in living systems, i.e., isolated from other proteins, etc. Any of the peptides provided herein may be isolated.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheriodal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 1000:1, still preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to aquire a stable and reproducible distribution based on appropriate inputs for viscosity of the medium and refractive indicies of the sample. The effective diameter, or mean of the distribution, is then reported.

“Obtained” means taken from a source without substantial modification. Substantial modification is modification that significantly affects the chemical or immunological properties of the material in question. For example, as a non-limiting example, a peptide or nucleic acid with a sequence with greater than 90%, preferably greater than 95%, preferably greater than 97%, preferably greater than 98%, preferably greater than 99%, preferably 100%, identity to a natural peptide or nucleotide sequence, preferably a natural consensus peptide or nucleotide sequence, and chemical and/or immunological properties that are not significantly different from the natural peptide or nucleic acid would be said to be obtained from the natural peptide or nucleotide sequence. These chemical or immunological properties comprise hydrophilicity, stability, affinity, and ability to couple with a carrier such as a synthetic nanocarrier.

“Peptide” means a compound comprising between 2 and 100 amino acids. Peptides according to the invention may be obtained or derived from a variety of sources, preferably from human influenza A virus hemagglutinin.

“Pharmaceutically acceptable excipient” means a pharmacologically inactive material used together with the recited synthetic nanocarriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, inventive synthetic nanocarriers do not comprise chitosan. In embodiments, synthetic nanocarriers are present in an amount sufficient to provide an immune response to the peptide upon administration of the composition to a subject. In embodiments, amounts of the synthetic nanocarriers may range from 0.1 micrograms to 500 micrograms, preferably from 1 micrograms to 100 micrograms.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., or (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in WO2010047839A1 or WO2009106999A2, or (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural adjuvant (meaning that the VLPs comprise an adjuvant other than naturally occurring RNA generated during the production of the VLPs). In embodiments, synthetic nanocarriers may possess an aspect ratio greater than 1:1, 1:1.2, 1:1.5, 1:2, 1:3, 1:5, 1:7, or greater than 1:10.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cell antigens generally are proteins or peptides. T cell antigens may be an antigen that stimulates a CD8+ T cell response, a CD4+ T cell response, or both. The nanocarriers, therefore, in some embodiments can effectively stimulate both types of responses.

In some embodiments the T cell antigen is a T helper cell antigen (i.e. one that can generate an enhanced response to a B cell antigen, preferably an unrelated B cell antigen, through stimulation of T cell help). In embodiments, a T helper cell antigen may comprise one or more peptides obtained or derived from tetanus toxoid, Epstein-Barr virus, influenza virus, respiratory syncytial virus, measles virus, mumps virus, rubella virus, cytomegalovirus, adenovirus, diphtheria toxoid, or a PADRE peptide (known from the work of Sette et al. U.S. Pat. No. 7,202,351). In other embodiments, a T helper cell antigen may comprise one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as a T helper cell antigen, see V. Cerundolo et al., “Harnessing invariant NKT cells in vaccination strategies.” Nature Rev Immun, 9:28-38 (2009). In embodiments, CD4+ T-cell antigens may be derivatives of a CD4+ T-cell antigen that is obtained from a source, such as a natural source. In such embodiments, CD4+ T-cell antigen sequences, such as those peptides that bind to MHC II, may have at least 70%, 80%, 90%, or 95% identity to the antigen obtained from the source. In embodiments, the T cell antigen, preferably a T helper cell antigen, may be coupled to, or uncoupled from, a synthetic nanocarrier.

“Vaccine” means a composition of matter that improves the immune response to a particular pathogen or disease. A vaccine typically contains factors that stimulate a subject's immune system to recognize a specific antigen as foreign and eliminate it from the subject's body. A vaccine also establishes an immunologic ‘memory’ so the antigen will be quickly recognized and responded to if a person is re-challenged. Vaccines can be prophylactic (for example to prevent future infection by any pathogen), or therapeutic (for example a vaccine against a tumor specific antigen for the treatment of cancer). In embodiments, a vaccine may comprise dosage forms according to the invention.

### III. Inventive Compositions

In certain embodiments, the invention encompasses certain derivatives of an A-helix of an epitope on HA2 subunit of human influenza A virus hemagglutinin that is bound by antibody CR6261. The A-helix is believed to be a primary site of interaction by antibody CR6261 via formation of multiple hydrogen bonds (e.g., by Gln42, Asp46, Thr49 and Asn53 in the 1918/H1 strain) as well as several hydrophobic bonds (e.g., by Thr41, Ile45, Thr49, Va152, and Ile56 in 1918/H1 strain). Furthermore, these interactions are known to be crucial for conserved recognition of different influenza strains (e.g., H5/Vietnam) by antibody CR6261 (Ekiert et al., Science; 324: 246-251, 2009).

While the α-helical structure of the A-Helix is maintained by the entire protein sequence, a short sequence peptide present in an aqueous environment often forms a random coil structure, due to the competition of the H-bonding formation when exposed to the aqueous environment. Modifications to the A-helix were introduced with the intent of increasing the propensity of the A-helix to maintain its α-helical structure upon exposure to aqueous environments, thus preserving the antibody contacting epitope in the appropriate conformation. Approaches included (i) reducing conformation constraint, (ii) increasing sequence length and hydrophobicity at sequence end to stabilize the intra-molecular H-bonding, (iii) introducing ionic interaction (salt-bridge formation) or π-π stacking to stabilize the existing intra-molecular H-bonding. Further modifications of the peptide may include addition of two C-terminal glycines with a linking group such as an acetylene group at the C-terminus to enable efficient coupling of the peptide to carriers such as synthetic nanocarriers while maintaining its natural conformation, and also addition of an acetyl group at the N-terminus of the peptide to enable better peptide exposure to antibodies (preventing the N-terminus from “looping back” to nanocarrier surface).

In one embodiment of the invention, amino acid residues within HA-based peptide corresponding to positions 39, 43, 48, 51 and 55 (mostly polar residues residing on the side of helix that is opposite to antibody-binding site) were changed to alanines to reduce the conformation constrain (SEQ HA2),

| | |
|---|---|
| | (SEQ ID NO: 11) |
| SEQ HA1: | AADKESTQKAIDGVTNKVNSIIDKGG-propargyl |
| | (SEQ ID NO: 12) |
| SEQ HA2: | 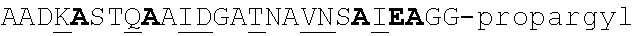 |

Additional modifications can be made to the inventive peptides, according to the formula:

(SEQ ID NO: 1)
Acetyl-X₁KE X₂QKAID X₃TN X₄VN X₅I X₆ X₇-R

where
   X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
   X₂=ST or AA
   X₃=GV or AA
   X₄=K or A
   X₅=SI, SA or AA
   X₆=DK, EA, DA, AA
   X₇=GG, GNG (SEQ ID NO: 10)
   R═COOH, or a linking group for coupling to a carrier such as a synthetic nanocarrier.

In some cases, the C-terminus amino acid sequence GG which is added for the efficient coupling of the propargyl linker is altered to GNG. In some other embodiments, similar to the approach made to X₁, additional amino acid sequences are also added to the C-terminus to increase the hydrophobicity of the peptide. The amino acid sequences added are ANAA (SEQ ID NO: 6), ANALLI (SEQ ID NO: 7), ANLLI (SEQ ID NO: 8), WNAAWG (SEQ ID NO: 9).

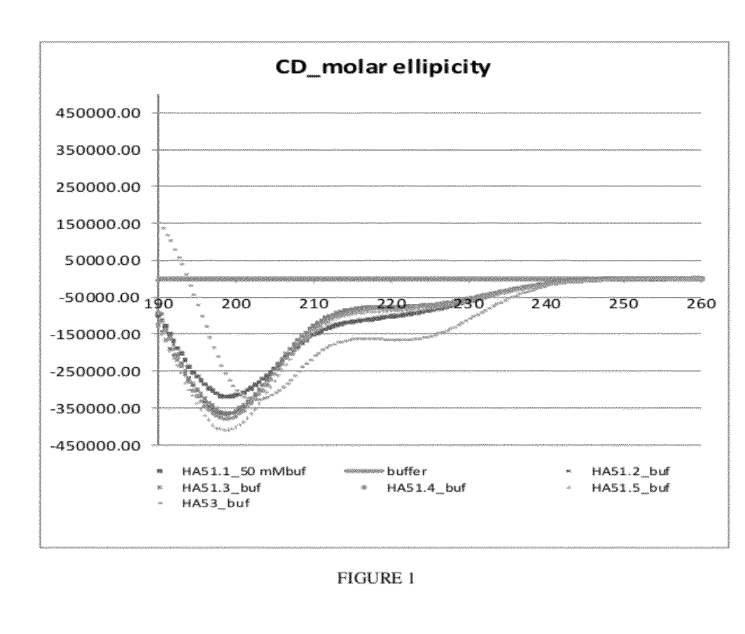

Secondary structure of the peptides were measured by circular dichroism to evaluate the design. The various modifications were effective in encouraging the intended α-helical structure to different degrees. For certain sequences (ex. SEQ HA1, A2, HA51.1, HA51.2, HA51.3, HA51.4, see FIG. 1), the lack of 208 nm and 222 nm double-dips signal which is indicative of the rich helical content structure and negative signal at the 190-200 nm region suggests these peptides are less optimal for forming α-helical structures in aqueous environments. An improvement of the α-helical structure content is observed for peptide SEQ HA 53, where the peptide is designed with longer sequences (X₁=AADAAD (SEQ ID NO: 3), along with replacement of alanine to the positions at X₂, X₃, X₅ and X₆).

FIG. 1 shows circular dichroic measurements of peptides according to the invention.

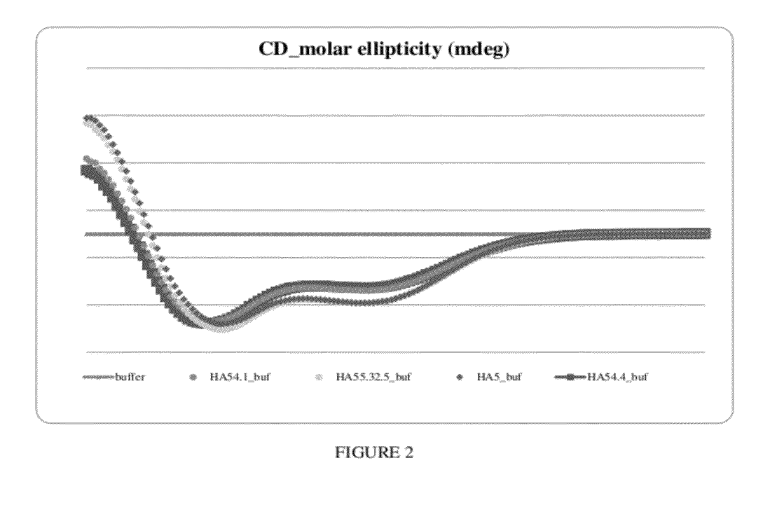

More significant improvement of the α-helical content of the peptide structure was observed for peptides, SEQHAS, HA55.32.4, HA54.1, and HA54.4. See FIG. 2. FIG. 2 shows circular dichroic measurements of additional peptides according to the invention.

TABLE 1

| | SEQ ID NO |
|---|---|
| 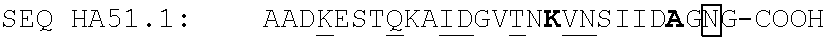 | (13) |
| 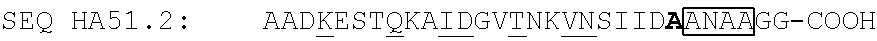 | (14) |
| 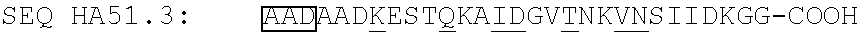 | (15) |
| 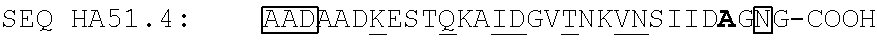 | (16) |
| 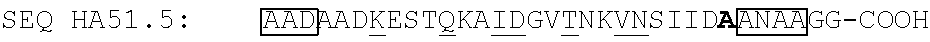 | (17) |
| 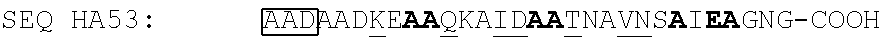 | (18) |
| 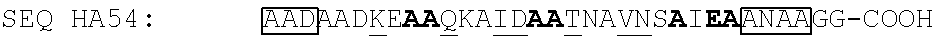 | (19) |
| 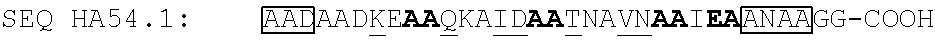 | (20) |
| 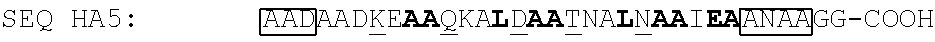 | (21) |
| 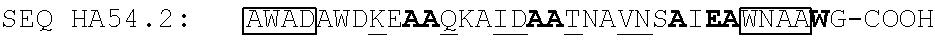 | (22) |
| 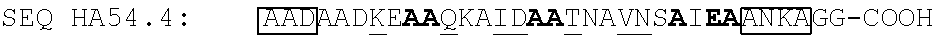 | (23) |
| 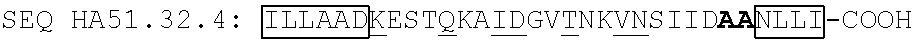 | (24) |
| 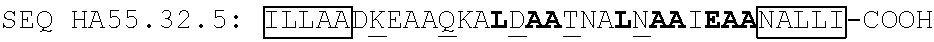 | (25) |

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.). In some embodiments, various elements of the synthetic nanocarriers can be coupled with the polymer.

In some embodiments, an immunofeature surface, targeting moiety, and/or oligonucleotide (or other element) can be covalently associated with a polymeric matrix. In some embodiments, covalent association is mediated by a linker. In some embodiments, an immunofeature surface, targeting moiety, and/or oligonucleotide (or other element) can be noncovalently associated with a polymeric matrix. For example, in some embodiments, an immunofeature surface, targeting moiety, and/or oligonucleotide (or other element) can be encapsulated within, surrounded by, and/or dispersed throughout a polymeric matrix. Alternatively or additionally, an immunofeature surface, targeting moiety, and/or nucleotide (or other element) can be associated with a polymeric matrix by hydrophobic interactions, charge interactions, van der Waals forces, etc.

A wide variety of polymers and methods for forming polymeric matrices therefrom are known conventionally. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5543158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol;sphingomyelin; phosphatidylethanolamine(cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the inventive synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

Compositions according to the invention comprise inventive synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. Inventive compositions may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

In embodiments, when preparing synthetic nanocarriers as carriers for adjuvants for use in vaccines, methods for coupling the adjuvants to the synthetic nanocarriers may be useful. If the adjuvant is a small molecule it may be of advantage to attach the adjuvant to a polymer prior to the assembly of the synthetic nanocarriers. In embodiments, it may also be an advantage to prepare the synthetic nanocarriers with surface groups that are used to couple the adjuvant to the synthetic nanocarrier through the use of these surface groups rather than attaching the adjuvant to a polymer and then using this polymer conjugate in the construction of synthetic nanocarriers.

Peptides can be coupled to the synthetic nanocarriers by a variety of methods. In embodiments, the peptide is coupled to an external surface of the synthetic nanocarrier covalently or non-covalently, preferably though its C terminus or its N-terminus, more preferably the peptide is coupled through its C terminus to the external surface.

In certain embodiments, the coupling can be a covalent linker. In embodiments, peptides according to the invention can be covalently coupled to the external surface via a 1,2,3-triazole linker formed by the 1,3-dipolar cycloaddition reaction of azido groups on the surface of the nanocarrier with peptides containing an alkyne group or by the 1,3-dipolar cycloaddition reaction of alkynes on the surface of the nanocarrier with peptides containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as the peptide with the carboxylic acid group of a second component such as the nanocarrier. The amide bond in the linker can be made using any of the conventional amide bond forming reactions with suitably protected amino acids or peptides and activated carboxylic acid such N-hydroxysuccinimide-activated ester.

A disulfide linker is made via the formation of a disulfide (S—S) bond between two sulfur atoms of the form, for instance, of R₁—S—S—R₂. A disulfide bond can be formed by thiol exchange of a peptide containing thiol/mercaptan group (—SH) with another activated thiol group on a polymer or nanocarrier or a nanocarrier containing thiol/mercaptan groups with a peptide containing activated thiol group.

A triazole linker, specifically a 1,2,3-triazole of the form

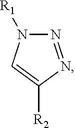

wherein R₁ and R₂ may be any chemical entities, is made by the 1,3-dipolar cycloaddition reaction of an azide attached to a first component such as the nanocarrier with a terminal alkyne attached to a second component such as the peptide. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The peptide is prepared with the presence of either an alkyne (if the polymer contains an azide) or an azide (if the polymer contains an alkyne) group. The peptide is then allowed to react with the nanocarrier via the 1,3-dipolar cycloaddition reaction with or without a catalyst which covalently couples the peptide to the particle through the 1,4-disubstituted 1,2,3-triazole linker.

A thioether linker is made by the formation of a sulfur-carbon (thioether) bond in the form, for instance, of R₁—S—R₂. Thioether can be made by either alkylation of a thiol/mercaptan (—SH) group on one component such as the peptide with an alkylating group such as halide or epoxide on a second component such as the nanocarrier. Thioether linkers can also be formed by Michael addition of a thiol/mercaptan group on one component such as a peptide to an electron-deficient alkene group on a second component such as a polymer containing a maleimide group or vinyl sulfone group as the Michael acceptor. In another way, thioether linkers can be prepared by the radical thiol-ene reaction of a thiol/mercaptan group on one component such as a peptide with an alkene group on a second component such as a polymer or nanocarrier.

A hydrazone linker is made by the reaction of a hydrazide group on one component such as the peptide with an aldehyde/ketone group on the second component such as the nanocarrier.

A hydrazide linker is formed by the reaction of a hydrazine group on one component such as the peptide with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component such as the peptide with an aldehyde or ketone group on the second component such as the nanocarrier.

An urea or thiourea linker is prepared by the reaction of an amine group on one component such as the peptide with an isocyanate or thioisocyanate group on the second component such as the nanocarrier.

An amidine linker is prepared by the reaction of an amine group on one component such as the peptide with an imidoester group on the second component such as the nanocarrier.

An amine linker is made by the alkylation reaction of an amine group on one component such as the peptide with an alkylating group such as halide, epoxide, or sulfonate ester group on the second component such as the nanocarrier. Alternatively, an amine linker can also be made by reductive amination of an amine group on one component such as the peptide with an aldehyde or ketone group on the second component such as the nanocarrier with a suitable reducing reagent such as sodium cyanoborohydride or sodium triacetoxyborohydride.

A sulfonamide linker is made by the reaction of an amine group on one component such as the peptide with a sulfonyl halide (such as sulfonyl chloride) group on the second component such as the nanocarrier.

A sulfone linker is made by Michael addition of a nucleophile to a vinyl sulfone. Either the vinyl sulfone or the nucleophile may be on the surface of the nanoparticle or attached to the antigen.

Additional descriptions of available conjugation methods are available in “Bioconjugate Techniques”, 2nd Edition By Greg T. Hermanson, Published by Academic Press, Inc., 2008) (Hermanson 2008.)

The peptide can also be conjugated to the nanocarrier via non-covalent conjugation methods. For examples, a negative charged peptide can be conjugated to a positive charged nanocarrier through electrostatic adsorption. A peptide containing a metal ligand can also be conjugated to a nanocarrier containing a metal complex via a metal-ligand complex.

In embodiments, an antigen can be coupled to a polymer, for example polylactic acid-block-polyethylene glycol, prior to the assembly of the synthetic nanocarrier or the synthetic nanocarrier can be formed with reactive or activatible groups on its surface. In the latter case, the peptide is prepared with a group that is compatible with the attachment chemistry that is presented by the synthetic nanocarriers' surface. In other embodiments, a peptide antigen can be coupled to VLPs or liposomes using a suitable linker. A linker is a compound or reagent that capable of coupling two molecules together. In an embodiment, the linker can be a homobifuntional or heterobifunctional reagent as described in Hermanson 2008. For example, a VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then coupled to a peptide antigen containing an acid group via the other end of the ADH linker on NC to produce the corresponding VLP or liposome peptide conjugate.

In the present embodiments, a peptide obtained or derived from HA protein according to the invention that comprises a C-terminal alkyne group may be coupled via the Cu(I)-catalyzed 1,3-dipolar cycloaddition (CuAAC) to synthetic nanocarriers made of PLA-PEG-azide polymer while the azide groups are on the surface of the synthetic nanocarriers. In a specific embodiment, the Cu(I) catalyst is formed in situ from CuSO4 and sodium ascorbate. Preferably, a suitable Cu(I) ligand such as Tris(3-hydroxypropyltriazolylmethyl)amine, is used to maintain the activity of the Cu(I) catalyst. The reaction is performed in buffered aq solution (pH 6-9) at 4 to 25 C over 2-48 h.

In addition to covalent attachment the peptide can be adsorbed to a pre-formed synthetic nanocarrier or it can be encapsulated during the formation of the synthetic nanocarrier.

In embodiments, the inventive synthetic nanocarriers may be coupled to one or more adjuvants, and/or may be coupled to a T-helper antigen. Types of adjuvants and T-helper antigen antigens useful in the practice of the present invention have been described elsewhere. The amounts of such adjuvants and/or T-helper antigen antigens to be included in the inventive synthetic nanocarriers may be determined using conventional dose ranging techniques. Adjuvants and/or T-helper antigen antigens may be coupled to the synthetic nanocarriers using coupling methods disclosed elsewhere herein, or known conventionally, and adapted for use with the particular adjuvant and/or T-helper antigen antigen (e.g. use of linker chemistries noted for use with the recited polypeptides, including the techniques of Hermanson 2008, or non-covalent coupling techniques (encapsulation, adsorption, and the like), etc., in each case adapted to the adjuvant and/or T-helper antigen antigen of interest may also be used). Use of adjuvants and/or T-helper antigen antigens can provide an improved immune response to the recited peptides.

### IV. Methods of Making and Using the Inventive Compositions and Related Methods

Synthetic nanocarriers may be prepared using a wide variety of methods known in the art. For example, synthetic nanocarriers can be formed by methods as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755, and also US Patents 5578325 and 6007845) ; P. Paolicelli et al., “Surface-modified PLGA-based Nanoparticles that can Efficiently Associate and Deliver Virus-like Particles” Nanomedicine. 5(6):843-853 (2010)).

Various materials may be coupled through encapsulation into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based

Nanoparticles that can Efficiently Associate and Deliver Virus-like Particles” Nanomedicine. 5(6):843-853 (2010)). Other methods suitable for encapsulating materials, such as oligonucleotides, into synthetic nanocarriers may be used, including without limitation methods disclosed in U.S. Pat. No. 6,632,671 to Unger (Oct. 14, 2003).

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the inventive synthetic nanocarriers—such as moieties of which an immunofeature surface is comprised, targeting moieties, polymeric matrices, antigens and the like—may be coupled to the overall synthetic nanocarrier, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be coupled to immunofeature surfaces, targeting moieties, adjuvants, various antigens, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In embodiments, encapsulation and/or absorption are forms of coupling.

Doses of dosage forms contain varying amounts of synthetic nanocarriers and varying amounts of antigens, according to the invention. The amount of synthetic nanocarriers and/or antigens present in the inventive dosage forms can be varied according to the nature of the antigens, the therapeutic benefit to be accomplished, and other such parameters. In embodiments, dose ranging studies can be conducted to establish optimal therapeutic amount of the synthetic nanocarriers and the amount of peptide antigens to be present in the dosage form. In embodiments, the synthetic nanocarriers and the peptide antigens are present in the dosage form in an amount effective to generate an immune response to the peptide antigens upon administration to a subject. It is possible to determine amounts of the peptides effective to generate an immune response using conventional dose ranging studies and techniques in subjects. Inventive dosage forms may be administered at a variety of frequencies. In an embodiment, at least one administration of the dosage form is sufficient to generate a pharmacologically relevant response. In additional embodiments, at least two administrations, at least three administrations, or at least four administrations, of the dosage form are utilized to ensure a pharmacologically relevant response.

In embodiments, the inventive synthetic nanocarriers can be combined with other adjuvants by admixing in the same vehicle or delivery system. Such adjuvants may include, but are not limited to mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (ASO4), MPL A of above-mentioned bacteria separately, saponins, such as QS-21,Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In embodiments, the inventive synthetic nanocarriers can be combined with an antigen different, similar or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying synthetic nanocarrier administered separately at a different time-point and/or at a different body location and/or by a different immunization route. In some embodiments, such antigen is another influenza antigen, for example, a neuraminidase, a surface antigen, a nucleocapsid protein, a matrix protein, a phosphoprotein, a fusion protein, a hemagglutinin, a hemagglutinin-neuraminidase, a glycoprotein capsular polysaccharides, a protein D, a M2 protein, or an antigenic fragment thereof, of an influenza virus.

In embodiments, the inventive dosage forms may comprise the recited synthetic nanocarriers and one or more conventional influenza vaccines to form a multivalent influenza vaccine. This may be accomplished by simply admixing a dispersion comprising the recited synthetic nanocarriers with a solution or dispersion that comprises a conventional influenza vaccine. In an embodiment, the inventive dosage forms comprise the recited synthetic nanocarriers and influenza antigen that is not coupled to the recited synthetic nanocarriers. Such conventional influenza vaccines include, for example,

Populations of synthetic nanocarriers may be combined to form pharmaceutical dosage forms according to the present invention using traditional pharmaceutical mixing methods. These include liquid-liquid mixing in which two or more suspensions, each containing one or more subset of nanocarriers, are directly combined or are brought together via one or more vessels containing diluent. As synthetic nanocarriers may also be produced or stored in a powder form, dry powder-powder mixing could be performed as could the re-suspension of two or more powders in a common media. Depending on the properties of the nanocarriers and their interaction potentials, there may be advantages conferred to one or another route of mixing.

Compositions according to the invention comprise inventive synthetic nanocarriers in combination with pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, inventive synthetic nanocarriers are suspended in sterile saline solution for injection together with a preservative.

It is to be understood that the compositions of the invention can be made in any suitable manner, and the invention is in no way limited to compositions that can be produced using the methods described herein. Selection of an appropriate method may require attention to the properties of the particular moieties being associated.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to intravenous, subcutaneous, pulmonary, intramuscular, intradermal, oral, intranasal, intramucosal, transmucosal, sublingual, rectal; ophthalmic, transdermal, transcutaneous or by a combination of these routes.

The compositions and methods described herein can be used to induce, enhance, suppress, modulate, direct, or redirect an immune response. The compositions and methods described herein can be used in the diagnosis, prophylaxis and/or treatment of conditions such as human influenza infections or other related disorders and/or conditions.

### EXAMPLES

### Example 1

### Synthetic Nanocarriers with Covalently Coupled Peptides from Human Influenza A Virus Hemagglutinin

Modified HA peptides (HAP) containing a terminal alkyne linker were conjugated to the synthetic nanocarriers containing surface azide groups via a 1,4-triazole linker formed by the copper-catalyzed 1,3-dipolar cycloaddition reaction (CuAAC or click reaction) as described below:

PLGA-R848 was prepared by reaction of PLGA polymer containing acid end group with R848 in the presence of coupling agent such as HBTU as follows:

A mixture of PLGA (Lakeshores Polymers, MW ˜5000, 7525DLG1A, acid number 0.7 mmol/g, 10 g, 7.0 mmol) and HBTU (5.3 g, 14 mmol) in anhydrous EtOAc (160 mL) was stirred at room temperature under argon for 50 minutes. Compound R848 (2.2 g, 7 mmol) was added, followed by diisopropylethylamine (DIPEA) (5 mL, 28 mmol). The mixture was stirred at room temperature for 6 h and then at 50-55° C. overnight (about 16 h). After cooling, the mixture was diluted with EtOAc (200 mL) and washed with saturated NH₄Cl solution (2×40 mL), water (40 mL) and brine solution (40 mL). The solution was dried over Na₂SO₄ (20 g) and concentrated to a gel-like residue. Isopropyl alcohol (IPA) (300 mL) was then added and the polymer conjugate precipitated out of solution. The polymer was then washed with IPA (4×50 mL) to remove residual reagents and dried under vacuum at 35-40° C. for 3 days as a white powder (10.26 g, MW by GPC is 5200, R848 loading is 12% by HPLC).

PLA-PEG-N3 polymer was prepared by ring opening polymerization of HO-PEG-azide with dl-lactide in the presence of a catalyst such as Sn(Oct)2 as follows:

HO-PEG-CO2H (MW 3500, 1.33 g, 0.38 mmol) was treated with NH2-PEG3-N3 (MW 218.2, 0.1 g, 0.458 mmol) in the presence of DCC (MW 206, 0.117 g, 0.57 mmol) and NHS (MW 115, 0.066 g, 0.57 mmol) in dry DCM (10 mL) overnight. After filtration to remove insoluble byproduct (DCC-urea), the solution was concentrated and then diluted with ether to precipitate out the polymer, HO-PEG-N3 (1.17 g). After drying, HO-PEG-N3 (MW 3700, 1.17 g, 0.32 mmol) was mixed with dl-lactide (recrystallized from EtOAc, MW 144, 6.83 g, 47.4 mmol) and Na2SO4 (10 g) in a 100 mL flask. The solid mixture was dried under vacuum at 45 C overnight and dry toluene (30 mL) was added. The resulting suspension was heated to 110 C under argon and Sn(Oct)2 (MW 405, 0.1 mL, 0.32 mmol) was added. The mixture was heated at reflux for 18 h and cooled to rt. The mixture was diluted with DCM (50 mL) and filtered. After concentration to an oily residue, MTBE (200 mL) was added to precipitate out the polymer which was washed once with 100 mL of 10% MeOH in MTBE and 50 mL of MTBE. After drying, PLA-PEG-N3 was obtained as a white foam (7.2 g, average MW: 23,700 by H NMR).

Synthetic nanocarriers (NC) made up of PLGA-R848, PLA-PEG-N3 (linker to peptide antigen) and ova peptide (T-helper antigen) were prepared via a double emulsion method wherein the ova peptide (ova (323-339), sequence: H-Ile-Ser-Gln-Ala-Val-His-Ala-Ala-His-Ala-Glu-Ile-Asn-Glu-Ala-Gly-Arg-NH2 (SEQ ID NO: 26), acetate salt, Lot#B06395, prepared by Bachem Biosciences, Inc.) was encapsulated in the NCs. To a suspension of the NCs (9.5 mg/mL in PBS (pH 7.4 buffer), 1.85 mL, containing about 4.4 mg (MW: 25,000; 0.00018 mmol, 1.0 eq) of PLA-PEG-N3) was added modified HAP1 peptide containing an alkyne linker (sequence: Acetyl-Ala-Ala-Asp-Lys-Glu-Ser-Thr-Gln-Lys-Ala-Ile-Asp-Gly-Val-Thr-Asn-Lys-Val-Asn-Ser-Ile-Ile-Asp-Lys-Gly-Gly-NHCH2CCH (SEQ ID NO: 27) (C-terminal glycine propargyl amide) as acetate salt; Lot No. B06545 (prepared by Bachem Biosciences, Inc.); MW 2739; 2 eq, 0.00036 mmol, 1 mg) with gentle stirring. A solution of CuSO4 (20 mM in H2O, 0.02 mL) and a solution of copper (I) ligand, Tris(3-hydroxypropyltriazolylmethyl)amine (THPTA) (100 mM in H2O, 0.02 mL) were mixed and the resulting solution was added to the nanocarrier suspension. A solution of aminoguanidine hydrochloride salt (200 mM in H2O, 0.05 mL) was added, followed by a solution sodium ascorbate (200 mM in H2O, 0.05 mL). The resulting suspension was stirred at room temperature in the dark for 18 h. The suspension was then diluted with PBS buffer (pH 7.4) to 3 mL and centrifuged to remove the supernatant. The residual NC pellets were washed with 2×3 mL PBS buffer. The washed NC-HAP1 conjugates were then re-suspended in 2 mL of PBS buffer and stored frozen until further analysis and biological tests.

### Example 2

### Synthetic Nanocarriers with Covalently Coupled Peptide from Human Influenza A Virus Hemagglutinin

In a same fashion as Example -1, NC-HAP-2 conjugates were prepared as follows: Synthetic nanocarriers (NC) comprising PLGA-R848 (adjuvant), PLA-PEG-N3 (linker to peptide antigen), and ova peptide (T-cell antigen) were prepared via double emulsion method wherein the ova peptide was encapsulated in the NCs. To a suspension of the NCs (9.5 mg/mL in PBS (pH 7.4 buffer), 1.85 mL, containing about 4.4 mg (MW: 25,000; 0.00018 mmol, 1.0 eq) of PLA-PEG-N3 was added modified HAP2 peptide containing an alkyne linker (sequence: Acetyl-Ala-Ala-Asp-Lys-Ala-Ser-Thr-Gln-Ala-Ala-Ile-Asp-Gly-Ala-Thr-Asn-Ala-Val-Asn-Ser-Ala-Ile-Glu-Ala-Gly-Gly-NHCH2CCH (SEQ ID NO: 28) (C-terminal glycine propargyl amide) as acetate salt; Lot No. B06553 (prepared by Bachem Biosciences, Inc.); MW 2454; 2 eq, 0.00036 mmol, ca. 1 mg) with gentle stirring. A solution of CuSO4 (20 mM in H2O, 0.02 mL) and a solution of copper (I) ligand, Tris(3-hydroxypropyltriazolylmethyl)amine (THPTA) (100 mM in H2O, 0.02 mL) were mixed and the resulting solution was added to the NC suspension. A solution of aminoguanidine hydrochloride salt (200 mM in H2O, 0.05 mL) was added, followed by a solution sodium ascorbate (200 mM in H2O, 0.05 mL). The resulting suspension was stirred at rt in dark for 18 h. The suspension was then diluted with PBS buffer (pH 7.4) to 3 mL and centrifuged to remove the supernatant. The residual NC pellets were washed with 2×3 mL PBS buffer. The washed NC-HAP2 conjugates were then re-suspended in 2 mL of PBS buffer and stored frozen until further analysis and biological tests.

### Example 3

### Synthetic Nanocarriers with Covalently Coupled Peptides from Human Influenza A Virus Hemagglutinin

In a similar manner to Examples 1 and 2 above, the following peptides were conjugated to synthetic nanocarriers comprising PLGA-R848, PLA-PEG-N3 and ova peptide:

(HAP54.1, SEQ ID NO: 29)
Acetyl-AADAADKEAAQKAIDAATNAVNAAIEAANAAGG-NHCH₂CCH
(C-terminal glycine propargyl amide)
(HAP5, SEQ ID NO: 30)
Acetyl-AADAADKEAAQKALDAATNALNAAIEAANAAGG-NHCH₂CCH
(C-terminal glycine propargyl amide)
(HAP54.4, SEQ ID NO: 31)
Acetyl-AADAADKEAKQKAIDAATNAVNSAIEAANKAGG-NHCH₂CCH
(C-terminal glycine propargyl amide)
(HAP55.32.5, SEQ ID NO: 32)
Acetyl-ILLAADKEAAQKALDAATNALNAAIEAANALLI-NHCH₂CCH
(C-terminal glycine propargyl amide)

Thus, to a suspension of nanocarriers (consist of 25% w/w of PLA-PEG-N3, in PBS (7 mg/mL, 2 mL) was added one of the above peptides comprising an alkyne linker (1 mM final concentration in peptide). A solution of CuSO4 (100 mM in water, 0.04 mL) was added to a final concentration of 2 mM in CuSO4, followed by a freshly prepared sodium ascorbate solution in water (200 mM, 0.2 mL). The resulting suspension was stirred gently at 4 C overnight. The suspension was then diluted with PBS buffer (pH 7.4) to 5 mL and centrifuged to remove the supernatant. The residual nanocarrier pellets were washed with 2×5 mL PBS buffer. The washed nanocarrier-peptide conjugates were then re-suspended in 2 mL of PBS buffer and stored frozen until further analysis and biological tests.

### Example 4

### Synthetic Nanocarriers with Non-Covalently Coupled Peptides from Human Influenza A Virus Hemagglutinin and Based on an Ionic Complex Between an Acid and an Amine (Prophetic)

Synthetic nanocarriers having negative surface charges are prepared from PLGA-CO2H, PLGA-R848 and ova peptide in the presence of long chain alkyl sulfate such as sodium dodecylsulfate or sulfonated polymer such as sodium polystyrene sulfonate using standard synthetic nanocarrier synthesis methods such as nanoprecipitation or double-emulsion evaporation. The negatively charged synthetic nanocarriers are then coated with a positively charged HA peptide linked to polylysine via ionic interactions in an aqueous phase. The resulting synthetic nanocarriers are then suspended in PBS buffer as described above for further analysis and biological tests.

### Example 5

### Synthetic Nanocarriers with Covalently Coupled Peptides from Human Influenza A Virus Hemagglutinin Induce Antibody Response In Vivo

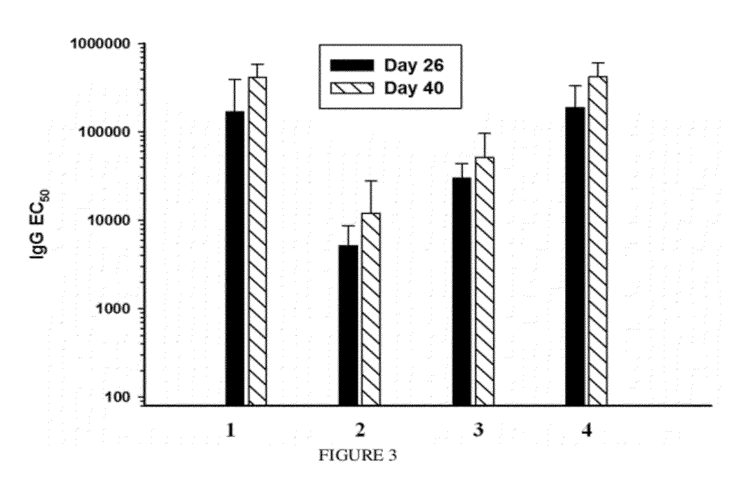

Synthetic nanocarriers (NC) containing conjugated adjuvant R848 (TLR7/8 agonist) and entrapped ovalbumin MHC class II peptide were covalently linked to two modified peptides HAP1 and HAP2 (amino acid sequences AADKESTQKAIDGVTNKVNSIIDKGG (SEQ ID NO: 33) and AADKASTQAAIDGATNAVNSAIEAGG (SEQ ID NO: 34), correspondingly) as illustrated in Example 1 (HAP1) and 2 HAP2,) respectively. These peptides mimic the conserved antigenic epitope present in the highly pathogenic strain A/Vietnam/1203/04(H5N1) A-helix of epitope CR6261 of the HA2 subunit of human influenza A virus hemagglutinin (particularly AA residues 35-58.) The immunization with these NC resulted in efficient generation of antibody responses comparable to or exceeding one induced by a purified HA protein. Specifically, antibody response induced by NC-HAP1 was markedly stronger than one induced by purified HA and equal to one induced by the mixture of HA with alum adjuvant (FIG. 3).

FIG. 3 shows anti-HAP antibody titers after NC-HAP vaccination (5 animals/group, subcutaneous route, 3 times, 2-wk interval). Groups 1 and 2: immunized with 100 μg of NC-HAP1 or NC-HAP2 (all NCs contained a combination of R848/Ova peptide), group 3: immunized with 1 μg of purified HA protein; group 4: immunized with 1 μg of purified HA in alum (1:1). Titers determined by ELISA against HAP1 (group 1), HAP2 (group 2) or against HA (groups 3 and 4). Titers for days 26 and 40 after the 1^{st} immunization are shown.

Synthetic nanocarriers (NC) containing conjugated adjuvant R848 (TLR7/8 agonist) and entrapped ovalbumin MHC class II peptide were covalently linked to four modified peptides HAP54.1, HAP5, HAP54.4, and HAP55.32.5 (amino acid sequences AADAADKEAAQKAIDAATNAVNAAIEAANAAGG (SEQ ID NO: 29), AADAADKEAAQKALDAATNALNAAIEAANAAGG (SEQ ID NO: 30), AADAADKEAKQKAIDAATNAVNSAIEAANKAGG (SEQ ID NO: 31), and ILLAADKEAAQKALDAATNALNAAIEAANALLI (SEQ ID NO: 32), correspondingly) as illustrated in example 3 (HAP54.1, HAP5, HAP54.4, and HAP55.32.5, respectively).

Nanocarriers were produced in a two-step process. First a base nanocarrier was formed with a reactive linkage site on the surface. Second the antigen was coupled to the linkage site on the nanocarrier surface by covalent reaction chemistry. The linkage chemistry in this example is the reaction of a terminal azide group on the nanocarrier with a terminal propargyl group on the peptide antigen.

### Materials:

Ovalbumin peptide 323-339 amide acetate salt, was purchased from Bachem Americas Inc. (3132 Kashiwa Street, Torrance Calif. 90505. Product code 4065609).

PLGA-R848, poly-D/L-lactide-co-glycolide, 4-amino-2-(ethoxymethyl)-α,α-dimethyl-1H-imidazo[4,5-c]quinoline-1-ethanol amide of approximately 5,200 Da made from PLGA of 3:1 lactide to glycolide ratio and having approximately 11.7% w/w conjugated resiquimod content was synthesized.

PLA-PEG-C6-Azide, block co-polymer consisting of a poly-D/L-lactide (PLA) block of approximately 23000 Da Mn and a polyethylene glycol (PEG) block of approximately 2000 Da Mn that is terminated by an azide functional group at the end of a linear six-carbon alkane, was synthesized from commercial starting materials by coupling HO-PEG-Acid to 6-azidohexan-1-amine, and then generating the PLA block by ring-opening polymerization of dl-lactide with the HO-PEG-C6-Azide.

PLA with an inherent viscosity of 0.21 dL/g was purchased from SurModics Pharmaceuticals (756 Tom Martin Drive, Birmingham, Ala. 35211. Product Code 100 DL 2A). Polyvinyl alcohol (Mw=11,000-31,000, 87-89% hydrolyzed) was purchased from J. T. Baker (Part Number U232-08).

### Step 1: Base Nanocarrier Production:

Solutions were prepared as follows: Solution 1: Ovalbumin peptide 323-339 at 20 mg/mL was prepared in 0.13N HCl at room temperature. Solution 2: PLGA-R848 at 50 mg/mL, PLA-PEG-C6-Azide at 25 mg/mL, and PLA at 25 mg/mL in dichloromethane was prepared by dissolving each polymer separately in dichloromethane at 100 mg/mL then combining 2 parts PLGA-R848 solution to 1 part PLA-PEG-C6-Azide solution to 1 part PLA solution. Solution 3: Polyvinyl alcohol @ 50 mg/mL in 100 mM in 100 mM phosphate buffer, pH 8. Solution 4: 70 mM phosphate buffer, pH 8.

A primary (W1/O) emulsion was first created using Solution 1 & Solution 2. Solution 1 (0.2 mL) and Solution 2 (1.0 mL) were combined in a small glass pressure tube, mixed by repeat pipetting to form a coarse dispersion, and sonicated at 50% amplitude for 40 seconds using a Branson Digital Sonifier 250.

A secondary (W1/O/W2) emulsion was then formed by adding Solution 3 (2.0 mL) to the primary emulsion, vortexing to create a coarse dispersion, and then sonicating at 30% amplitude for 40 seconds using the Branson Digital Sonifier 250.

The secondary emulsion was added to an open 50 mL beaker containing 70 mM phosphate buffer solution (30 mL) and stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 21,000 rcf for 45 minutes, removing the supernatant, and re-suspending the pellet in phosphate buffered saline. This washing procedure was repeated and then the pellet was re-suspended in phosphate buffered saline to achieve a nanocarrier suspension having a nominal concentration of 10 mg/mL on a polymer basis.

Two batches of nanocarriers were made in parallel, and then combined to form a single nanocarrier lot prior to analysis, storage, and use. The nanocarrier suspension was stored frozen at −20 C until use.

TABLE 2
Base Nanocarrier characterization

| Effective Diameter | | |
|---|---|---|
| by DLS (nm) | TLR Agonist, % w/w | T-cell agonist, % w/w |
| 246 | R848, 3.7 | Ova peptide 323-339, 2.0 |

### Step 2: Production of Antigen-Loaded Nanocarriers

HA-related peptides with a propargyl functional group at the C-terminus were purchased as custom products from CS Bio (Menlo Park, Calif.).

TABLE 3

| Peptide | Sequence |
|---|---|
| HA54.1 | Ac-Ala-Ala-Asp-Ala-Ala-Asp-Lys-Glu-Ala- |
| | Ala-Gln-Lys-Ala-Ile-Asp-Ala-Ala-Thr-Asn- |
| | Ala-Val-Asn-Ala-Ala-Ile-Glu-Ala-Ala-Asn- |
| | Ala-Ala-Gly-Gly-NH-CH2CCH (SEQ ID NO: 29) |
| HA5 | Ac-Ala-Ala-Asp-Ala-Ala-Asp-Lys-Glu-Ala- |
| | Ala-Gln-Lys-Ala-Leu-Asp-Ala-Ala-Thr-Asn- |
| | Ala-Leu-Asn-Ala-Ala-Ile-Glu-Ala-Ala-Asn- |
| | Ala-Ala-Gly-Gly-NH-CH2CCH (SEQ ID NO: 30) |
| HA54.4 | Ac-Ala-Ala-Asp-Ala-Ala-Asp-Lys-Glu-Ala- |
| | Gln-Lys-Ala-Ile-Asp-Ala-Ala-Thr-Asn-Ala- |
| | Val-Asn-Ser-Ala-Ile-Glu-Ala-Ala-Asn-Lys- |
| | Ala-Gly-Gly-NH-CH2CCH (SEQ ID NO: 31) |
| HA55.32.5 | Ac-Ile-Leu-Leu-Ala-Ala-Asp-Lys-Glu-Ala- |
| | Ala-Gln-Lys-Ala-Leu-Asp-Ala-Ala-Thr-Asn- |
| | Ala-Leu-Asn-Ala-Ala-Ile-Glu-Ala-Ala-Asn- |
| | Ala-Leu-Leu-Ile-NHCH2CCH (SEQ ID NO: 32) |

To couple the peptide to the nanocarrier, nanocarriers were first concentrated in phosphate-buffered saline (PBS) to approximately 23 mg/mL. Next the peptide was dissolved to make a clear 2 mM solution in PBS. The peptide and nanocarrier solutions were then combined and de-gassed with argon. 100 mM CuSO₄ and 200 mM tris(hydroxypropyltriazolyl)methylamine (THPTA) were then pre-mixed and added to the nanocarrier suspension, followed by sodium ascorbate (200 mM) to achieve final concentrations of 5, 10, and 20 mM respectively. The mixture was stirred at room temperature for 1 hour, refrigerated for 20 hours, and again at room temperature for 2 hours. The suspension was then diluted to 8 mL in PBS, pelleted to remove the supernatant, resuspended in 24 mL PBS, and re-pelleted. The nanocarriers were resuspended a final time at 5 mg/mL in sterile PBS.

These peptides mimic the conserved antigenic epitope present in the highly pathogenic strain A/Vietnam/1203/04(H5N1) alpha-helix of epitope CR2621 of the HA2 subunit of human influenza A virus hemagglutinin (particularly AA residues 35-58).

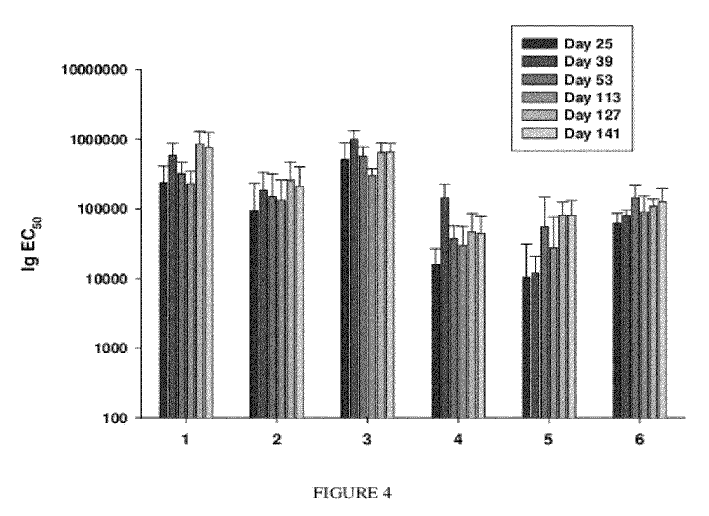

C57BL/6 mice were vaccinated using the synthetic nanocarriers (s.c., hind limbs, 60 μL total inoculation volume, 100 μg nanocarriers per injection, 3 times with a 2-week interval and 1 time at day 115). Group 1: immunized with 100 μg of NC-HAP54.1, group 2: immunized with 100 μg of NC-HAPS, group 3: immunized with 100 μg of NC-HAP54.4, group 4: immunized with 100 μg of NC-HAP55.32.5, group 5: immunized with 100 μg of NC-HAP2, group 6: immunized with 10 μg HAS protein plus alum (1:1). Mice were bled at days 25, 39, 53, 113, 127, and 141 and anti-HA peptide or anti-HA protein antibody titers were determined by a standard ELISA against HA peptide or HA protein (respective to the HA peptide or HA protein used for immunization). Results are shown in FIG. 4. Mice generated antibodies to all five of the HA peptides used with nanocarriers (HAP54.1, HAPS, HAP54.4, HAP55.32.5, and HAP2) at levels similar to or greater than those generated by HA protein with alum (FIG. 4).

FIG. 4 shows anti-HAP antibody titers after NC-HAP vaccination (5 animals/group, subcutaneous route, injected 3 times with a 2 week interval and once at day 115). Groups 1-5: immunized with 100 μg of NC-HAP54.1, NC-HAPS, NC-HAP54.4, NC-HAP55.32.5, or NC-HAP2, respectively; group 6: immunized with 10 μg of purified HA protein in alum (1:1). Titers determined by ELISA against HAP54.1 (group 1), HAPS (group 2), HAP54.4 (group 3), HAP55.32.5 (group 4), HAP2 (group 5) or HA protein (group 6). Titers for days 25, 39, 53, 113, 127, and 141 after the first immunization are shown.

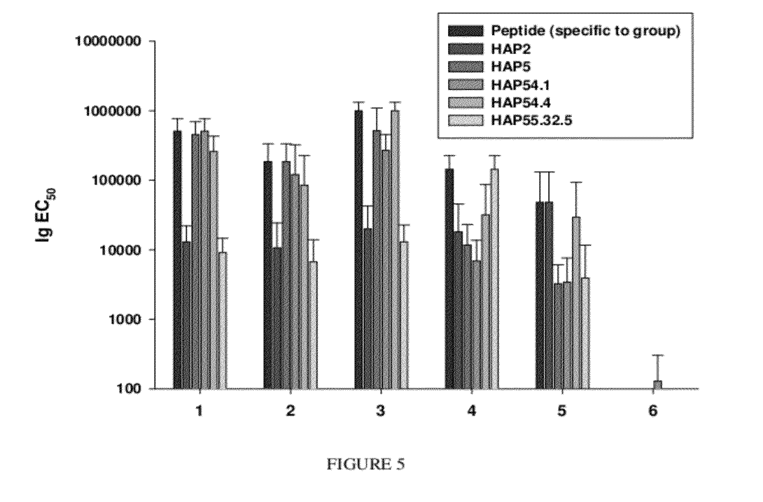

In addition, mice immunized with nanocarriers containing HA peptides (HAP54.1, HAPS, HAP54.4, HAP55.32.5, or HAP2) generated antibodies to the other HA peptides used with nanocarriers, indicating cross-protection across these sequences of HA peptides (FIG. 5). FIG. 5 shows anti-HAP antibody titers after NC-HAP vaccination (5 animals/group, subcutaneous route, injected 3 times with a 2 week interval and once at day 115). Groups 1-5: immunized with 100 μg of NC-HAP54.1, NC-HAPS, NC-HAP54.4, NC-HAP55.32.5, or NC-HAP2, respectively; group 6: immunized with 10 μg of purified HA protein in alum (1:1). Titers determined by ELISA against HAP54.1, HAPS, HAP54.4, HAP55.32.5, or HAP2. Titers for day 39 after the first immunization are shown.

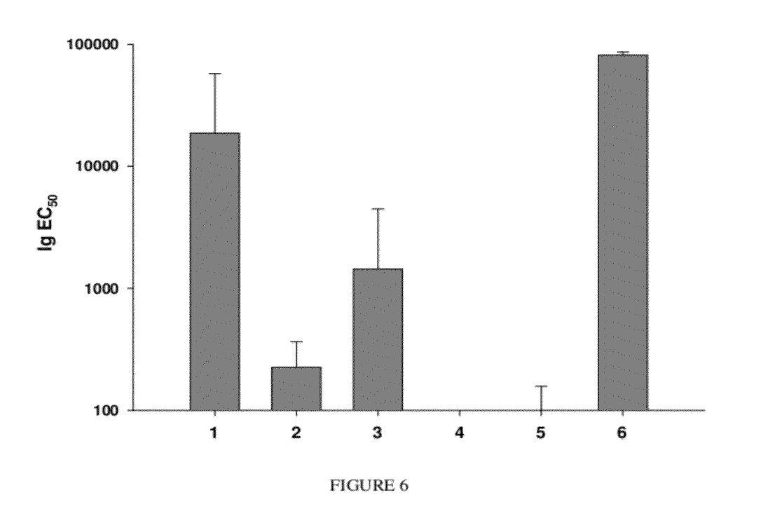

Mice immunized with NC-HAP54.1, NC-HAPS, or NC-HAP54.4 also generated antibodies that recognized H5N1 HA protein (FIG. 6). FIG. 6 shows anti-H5N1 HA protein antibody titers after NC-HAP vaccination (5 animals/group, subcutaneous route, injected 3 times with a 2 week interval and once at day 115). Groups 1-5: immunized with 100 μg of NC-HAP54.1, NC-HAPS, NC-HAP54.4, NC-HAP55.32.5, or NC-HAP2, respectively; group 6: immunized with 10 μg of purified HA protein in alum (1:1). Titers determined by ELISA against influenza virus H5N1 hemagglutinin protein. Titers for day 39 after the first immunization are shown.

Sera from immunized mice was collected and used to measure influenza viral neutralization using an HIV pseudovirus that expressed the H5N1 HA protein from the highly pathogenic avian influenza strain isolated in China A/Qinghai/59/05. All of the mice immunized with HA protein with alum generated neutralizing antibodies. Sera from four out of five mice in group 2, two out of five mice in group 3, and five out of five mice in group 4 showed neutralizing activity.

### REFERENCES

Black, M., Trent A., Tirrel M., and Olive, C. Advances in the design and delivery of peptide subunit vaccines with a focus on Toll-like receptor agonists. Expert Rev. Vaccines 2010; 9:157-173.

Caton, A. J., Brownlee, G. G., Yewdell, J. M. and Gerhard, W. The antigenic structure of the influenza virus A/PR/8/34 hemagglutinin (H1 subtype). Cell. 1982; 31:417-427.

Chakrabartty A., Kortemme T., Baldwin R. L. Helix propensities of the amino acids measured in alanine-based peptides without helix-stabilizing side-chain interactions. Protein Sci. 1994; 3:843-852.

Cross K. J., Langley W. A., Russell R. J., et al. Composition and functions of the influenza fusion peptide. Protein Pept. Lett. 2009; 16:766-778.

Ekiert D. C., Bhabha G., Elsliger M. A., et al. Antibody recognition of a highly conserved influenza virus epitope. Science 2009; 324:246-251.

Ellebedy A. H., Webby R. J. Influenza vaccines. Vaccine. 2009; 27 Suppl. 4:D65-8.

Jacchieri S. G. Richards N. G. Probing the influence of sequence-dependent interactions upon alpha-helix stability in alanine-based linear peptides. Biopolymers. 1993; 33:971-984.

Jimenez G. S., Planchon R., Wei Q., et al. Vaxfectin-formulated influenza DNA vaccines encoding NP and M2 viral proteins protect mice against lethal viral challenge. Hum. Vaccin. 2007; 3:157-164.

Kaverin N. V., Rudneva I. A., Ilyushina N. A., et al. Structure of antigenic sites on the haemagglutinin molecule of H5 avian influenza virus and phenotypic variation of escape mutants. J Gen Virol. 2002; 83:2497-2505.

Purcell, A. W., Zeng, W., Mifsud, N. A., et al. Dissecting the Role of Peptides in the

Immune Response: Theory, Practice and the Application to Vaccine Design. J. Peptide Sci. 2003; 9: 255-281.

Roose K., Fiers W., Saelens X. Pandemic preparedness: toward a universal influenza vaccine. Drug News Perspect. 2009; 22:80-92.

Sui J., Hwang W. C., Perez S., et al. Structural and functional bases for broad-spectrum neutralization of avian and human influenza A viruses. Nat Struct Mol Biol. 2009; 16:265-273.

Throsby M., van den Brink E., Jongeneelen M., et al. Heterosubtypic neutralizing monoclonal antibodies cross-protective against H5N1 and H1N1 recovered from human IgM+ memory B cells. PLoS One. 2008; 3:e3942.

Tsuchiya, E., Sugawara, K., Hongo, S., Matsuzaki, Y., Muraki, Y., Li, Z.-N. and Nakamura, K. Antigenic structure of the haemagglutinin of human influenza A/H2N2 virus. Journal of General Virology 2001; 82:2475-2484.

Wiley, D. C., Wilson, I. A., Skehel, J. J. Structural identification of the antibody-binding sites of Hong Kong influenza haemagglutinin and their involvement in antigenic variation. Nature. 1981; 289:373-378.

1. A dosage form comprising synthetic nanocarriers coupled to peptides that are obtained or derived from human influenza A virus hemagglutinin.

2. (canceled)

3. The dosage form of claim 1, wherein the peptides are obtained or derived from an HA1 subunit of human influenza A virus hemagglutinin or from an HA2 subunit of human influenza A virus hemagglutinin.

4. (canceled)

5. The dosage form of claim 3, wherein the peptides are obtained or derived from an A-helix of an epitope on HA2 subunit of human influenza A virus hemagglutinin that is bound by antibody CR6261.

6. The dosage form of claim 1, wherein the peptides obtained or derived from human influenza A virus hemagglutinin comprise a peptide of the formula:
(SEQ ID NO: 1)
Acetyl-X₁KE X₂QKAID X₃TN X₄VN X₅I X₆ X₇-R
where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10), and
R═COOH or a linking group for coupling to the synthetic nanocarriers.

7. The dosage form of claim 1, wherein the peptides comprise a peptide with an amino acid sequence as set forth in any one of SEQ ID NOs: 1, 11-25 and 27-34.

8-13. (canceled)

14. The dosage form of claim 1, wherein the synthetic nanocarriers are further coupled to one or more adjuvants.

15-16. (canceled)

17. The dosage form of claim 1, wherein the synthetic nanocarriers comprise lipid-based nanoparticles, polymeric nanoparticles, metallic nanoparticles, surfactant-based emulsions, dendrimers, buckyballs, nanowires, virus-like particles, peptide or protein-based particles, lipid-polymer nanoparticles, spheroidal nanoparticles, cubic nanoparticles, pyramidal nanoparticles, oblong nanoparticles, cylindrical nanoparticles, or toroidal nanoparticles.

18. (canceled)

19. The dosage form of claim 1, wherein the synthetic nanocarriers are further coupled to T-helper antigens.

20. (canceled)

21. The dosage form of claim 1, further comprising influenza antigen that is not coupled to the synthetic nanocarriers.

22. A method comprising administering the dosage form of claim 1 to a subject.

23-26. (canceled)

27. A method comprising:
providing synthetic nanocarriers; and
coupling peptides that are obtained or derived from human influenza A virus hemagglutinin to the synthetic nanocarriers.

28. (canceled)

29. A composition, dosage form or vaccine obtained, or obtainable, by a method as defined in claim 27.

30. A process for producing a composition, dosage form or vaccine comprising the steps of:
providing synthetic nanocarriers; and
coupling peptides that are obtained or derived from human influenza A virus hemagglutinin to the synthetic nanocarriers.

31. A dosage form comprising peptides obtained or derived from human influenza A virus hemagglutinin that generates in a subject polyclonal antibodies that compete for binding to human influenza A virus hemagglutinin with a control antibody, wherein the control antibody is CR6261.

32. (canceled)

33. The dosage form of claim 31, wherein the peptides are obtained or derived from an HA1 subunit of human influenza A virus hemagglutinin or from an HA2 subunit of human influenza A virus hemagglutinin.

34. (canceled)

35. The dosage form of claim 34, wherein the peptides are obtained or derived from an A-helix of an epitope on HA2 subunit of human influenza A virus hemagglutinin that is bound by antibody CR6261.

36. The dosage form of claim 31, wherein the peptides obtained or derived from human influenza A virus hemagglutinin comprise a peptide of the formula:
(SEQ ID NO: 1)
Acetyl-X₁KE X₂QKAID X₃TN X₄VN X₅I X₆ X₇-R
where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10), and
R═COOH or a linking group.

37-56. (canceled)

57. A method comprising administering the dosage form of claim 31 to a subject.

58-61. (canceled)

62. A composition comprising a peptide of the formula:
(SEQ ID NO: 1)
Acetyl-X₁KE X₂QKAID X₃TN X₄VN X₅I X₆ X₇-R
where X₁=AAD (SEQ ID NO: 2), AADAAD (SEQ ID NO: 3), AWADAWD (SEQ ID NO: 4), ILLAAD (SEQ ID NO: 5), ANAA (SEQ ID NO: 6), ANALLI (SEQ ID NO: 7), ANLLI (SEQ ID NO: 8), or WNAAWG (SEQ ID NO: 9)
X₂=ST or AA
X₃=GV or AA
X₄=K or A
X₅=SI, SA or AA
X₆=DK, EA, DA, or AA
X₇=GG, or GNG (SEQ ID NO: 10)
R═COOH or a linking group.

63. A composition comprising a peptide that has the amino acid sequence as set forth in SEQ ID NO: 1.

64-69. (canceled)


SEQUENCE LISTING (34 sequences)

SEQ ID NO: 1 - protein, 21 aa, ARTIFICIAL SEQUENCE
  synthetic polypeptide
KEXXQKAIDXXTNXVNXXIXX

SEQ ID NO: 2 - protein, 3 aa, ARTIFICIAL SEQUENCE
  synthetic polypeptide
AAD

SEQ ID NO: 3 - protein, 6 aa, ARTIFICIAL SEQUENCE
  synthetic polypeptide
AADAAD

SEQ ID NO: 4 - protein, 7 aa, ARTIFICIAL SEQUENCE
  synthetic polypeptide
AWADAWD

SEQ ID NO: 5 - protein, 6 aa, ARTIFICIAL SEQUENCE
  synthetic polypeptide
ILLAAD

SEQ ID NO: 6 - protein, 4 aa, ARTIFICIAL SEQUENCE
  synthetic polypeptide
ANAA

SEQ ID NO: 7 - protein, 6 aa, ARTIFICIAL SEQUENCE
  synthetic polypeptide
ANALLI

SEQ ID NO: 8 - protein, 5 aa, ARTIFICIAL SEQUENCE
  synthetic polypeptide
ANLLI

SEQ ID NO: 9 - protein, 6 aa, ARTIFICIAL SEQUENCE
  synthetic polypeptide
WNAAWG

SEQ ID NO: 10 - protein, 3 aa, ARTIFICIAL SEQUENCE
  synthetic polypeptide
GNG

SEQ ID NO: 11 - protein, 26 aa, ARTIFICIAL SEQUENCE
  synthetic polypeptide
AADKESTQKAIDGVTNKVNSIIDKGG

SEQ ID NO: 12 - protein, 26 aa, ARTIFICIAL SEQUENCE
  synthetic polypeptide
AADKASTQAAIDGATNAVNSAIEAGG

SEQ ID NO: 13 - protein, 27 aa, ARTIFICIAL SEQUENCE
  synthetic polypeptide
AADKESTQKAIDGVTNKVNSIIDAGNG

SEQ ID NO: 14 - protein, 30 aa, ARTIFICIAL SEQUENCE
  synthetic polypeptide
AADKESTQKAIDGVTNKVNSIIDAANAAGG

SEQ ID NO: 15 - protein, 29 aa, ARTIFICIAL SEQUENCE
  synthetic polypeptide
AADAADKESTQKAIDGVTNKVNSIIDKGG

SEQ ID NO: 16 - protein, 30 aa, ARTIFICIAL SEQUENCE
  synthetic polypeptide
AADAADKESTQKAIDGVTNKVNSIIDAGNG

SEQ ID NO: 17 - protein, 33 aa, ARTIFICIAL SEQUENCE
  synthetic polypeptide
AADAADKESTQKAIDGVTNKVNSIIDAANAAGG

SEQ ID NO: 18 - protein, 30 aa, ARTIFICIAL SEQUENCE
  synthetic polypeptide
AADAADKEAAQKAIDAATNAVNSAIEAGNG

SEQ ID NO: 19 - protein, 33 aa, ARTIFICIAL SEQUENCE
  synthetic polypeptide
AADAADKEAAQKAIDAATNAVNSAIEAANAAGG

SEQ ID NO: 20 - protein, 33 aa, ARTIFICIAL SEQUENCE
  synthetic polypeptide
AADAADKEAAQKAIDAATNAVNAAIEAANAAGG

SEQ ID NO: 21 - protein, 33 aa, ARTIFICIAL SEQUENCE
  synthetic polypeptide
AADAADKEAAQKALDAATNALNAAIEAANAAGG

SEQ ID NO: 22 - protein, 34 aa, ARTIFICIAL SEQUENCE
  synthetic polypeptide
AWADAWDKEAAQKAIDAATNAVNSAIEAWNAAWG

SEQ ID NO: 23 - protein, 33 aa, ARTIFICIAL SEQUENCE
  synthetic polypeptide
AADAADKEAAQKAIDAATNAVNSAIEAANKAGG

SEQ ID NO: 24 - protein, 32 aa, ARTIFICIAL SEQUENCE
  synthetic polypeptide
ILLAADKESTQKAIDGVTNKVNSIIDAANLLI

SEQ ID NO: 25 - protein, 33 aa, ARTIFICIAL SEQUENCE
  synthetic polypeptide
ILLAADKEAAQKALDAATNALNAAIEAANALLI

SEQ ID NO: 26 - protein, 17 aa, ARTIFICIAL SEQUENCE
  synthetic polypeptide
ISQAVHAAHAEINEAGR

SEQ ID NO: 27 - protein, 26 aa, ARTIFICIAL SEQUENCE
  synthetic polypeptide
AADKESTQKAIDGVTNKVNSIIDKGG

SEQ ID NO: 28 - protein, 26 aa, ARTIFICIAL SEQUENCE
  synthetic polypeptide
AADKASTQAAIDGATNAVNSAIEAGG

SEQ ID NO: 29 - protein, 33 aa, ARTIFICIAL SEQUENCE; identical to SEQ ID NO: 20
  synthetic polypeptide

SEQ ID NO: 30 - protein, 33 aa, ARTIFICIAL SEQUENCE; identical to SEQ ID NO: 21
  synthetic polypeptide

SEQ ID NO: 31 - protein, 33 aa, ARTIFICIAL SEQUENCE
  synthetic polypeptide
AADAADKEAKQKAIDAATNAVNSAIEAANKAGG

SEQ ID NO: 32 - protein, 34 aa, ARTIFICIAL SEQUENCE
  synthetic polypeptide
ILLAADKEAAQKALDAATNALNAAIEAANALLIX

SEQ ID NO: 33 - protein, 26 aa, ARTIFICIAL SEQUENCE
  synthetic polypeptide
AADKESTQKAIDGVTNKVNSIIDKGG

SEQ ID NO: 34 - protein, 26 aa, ARTIFICIAL SEQUENCE
  synthetic polypeptide
AADKASTQAAIDGATNAVNSAIEAGG